(19) United States
(12) Patent Application Publication
(10) Pub. No.: US 2007/0187243 A1
(43) Pub. Date: Aug. 16, 2007

(54) Planar electrochromatography/thin layer chromatography separations systems

(75) Inventors: Wayne F. Patton, Newton, MA (US); Venkateswarlu Panchagnula, Tarnaka Hyderabad (IN); Alvydas Mikulskis, West Roxbury, MA (US)

Correspondence Address: WILMER CUTLER PICKERING HALE AND DORR LLP, 60 STATE STREET, BOSTON, MA, 02109, US

(73) Assignee: PerkinElmer Life and Analytical Sciences

(21) Appl. No.: 11/595,234

(22) Filed: Nov. 10, 2006

Related U.S. Application Data
(60) Provisional application No. 60/735,326, filed on Nov. 10, 2005.

(51) Int. Cl. C07K 1/26; G01N 27/00

(52) U.S. Cl. 204/450; 204/600

(57) ABSTRACT

A method for separating a sample comprising a plurality of compounds includes loading a sample onto a planar stationary phase, said sample comprising a plurality of compounds to be separated; and, while retaining the sample on the planar stationary phase, subjecting the sample to planar electrochromatographic separation in a selected direction using a first mobile phase; and subjecting the sample to a chromatographically-based separation in a direction other than that of the direction used for planar electrochromatographic separation using a second mobile phase. Separation is optionally followed by direct detection of analytes using mass spectrometry (MS). These three dimensions of separation are orthogonal to one another, providing for improved resolution of the analytes.

### RELATED APPLICATIONS

This application claims the benefit of priority under 35 U.S.C. § 119(e) to co-pending U.S. application Ser. No. 60/735,326, filed Nov. 10, 2005, the contents of which are incorporated by reference.

### BACKGROUND

The human proteome is known to contain approximately 22,000 different genes, but due to posttranslational modifications and differential mRNA splicing, the total number of distinct proteins is most likely to be close to one million. The level of complexity, coupled with the relative abundances of different proteins, presents unique challenges in terms of separations technologies. Two-dimensional (2D) or even multi-dimensional protein separations are favored over single dimension separations in proteomics due to the increased resolution afforded by the additional dimensions of fractionation.

Two-dimensional separations systems use two different modes of separation to improve the separation of a complex mixture of compounds. The two different separations techniques typically rely on different physiochemical properties of the molecules to effect separation. For example, proteins can be initially separated on the basis of their molecular charge and then subsequently the 2nd dimension of separation takes advantage of the differences in size of various proteins. Under ideal circumstances the different dimensions of separation are orthogonal, that is, the separations are statistically independent.

D separation systems can be categorized by the type of interface between the dimensions. In “heart-cutting” methods a region of interest is selected from the first dimension and the selected region is subjected to second dimension separation. Systems that subject the entire first dimension to a second dimension separation, or alternatively sampling of the effluent from the first dimension at regular intervals and fixed volumes for subsequent fractionation in the second dimension, are referred to as “comprehensive” methods. A two-dimensional separation is considered comprehensive if every part of the sample is subjected to two separations and an equal portion of all sample components are subjected to two separations. Ideally, the degree of separation of the first dimension is essentially maintained during the second separation.

The analysis of the proteome typically require intact proteins to be digested into their constituent peptides and then fractionated by a combination of chromatographic and/or electrophoretic techniques, prior to their identification. A commonly used protein separation technology is high-resolution two-dimensional gel electrophoresis (2DGE). High resolution 2DGE involves the separation of proteins in the first dimension according to their charge by isoelectric focusing and in the second dimension according to their relative mobility by sodium dodecyl sulfate polyacrylamide gel electrophoresis. The technique is capable of simultaneously resolving thousands of polypeptides as a constellation pattern of spots. 2DGE can be used for the global analysis of metabolic processes such as protein synthesis, glycolysis, gluconeogenesis, nucleotide biosynthesis, amino acid biosynthesis, lipid metabolism and stress response. It is also used for the analysis of signal transduction pathways, with an ability to detect global changes in signaling events, as well as to differentiate between changes in protein expression and degradation from changes arising through post-translational modification.

There are challenges associated with certain 2DGE applications. For example, analysis using 2DGE produces a pattern of spots, each corresponding to one or more proteins, which can be difficult to interpret due to smudging. There are also difficulties associated with detecting low abundance proteins, relatively basic proteins, relatively hydrophobic proteins, higher molecular weight proteins and lower molecular weight proteins. Additionally, polyacrylamide gels are mechanically fragile and thus are susceptible to stretching and breaking during handling. While detection of proteins directly in gels with labeled antibodies or lectins has been accomplished, the approach is not generally applicable to every antigen and is relatively insensitive. Consequently, proteins are usually electrophoretically transferred to polymeric membranes before detecting proteins of interest. The polyacrylamide gel also poses difficulties in the identification of proteins by microchemical characterization techniques, such as mass spectrometry. In microchemical characterization techniques, gels must be macerated, gel buffers rinsed away, proteins incubated with proteolytic enzymes, and peptides selectively retrieved and concentrated using a reverse-phase column prior to identification.

Other 2D techniques for protein separations include the use of thin-layer electrophoresis (TLE), such as electrically-driven cellulose-based separation of proteins. Using this technique, proteins are separated in the first dimension by electrophoresis; and in the second dimension by standard thin film chromatography, such as thin-layer chromatography. In TLE, hydrophilic cellulose-based plates are utilized as the stationary phase and proteins do not significantly interact with the cellulose matrix. Because proteins interact minimally with the cellulose stationary phase in aqueous medium, once the applied current is removed, the separated proteins can diffuse, which results in loss of detectable sample. In addition, commonly used cellulose acetate membranes are considered fragile in many laboratory settings and the generated profiles of very hydrophilic proteins, such as certain proteins contained in urinary and serum proteins, have low resolution compared to those generated with polyacrylamide gels.

### SUMMARY OF THE INVENTION

Systems and methods are provided for a high resolution protein, peptide and glycan separation system that employs a solid phase support and simple combinations of organic and aqueous mobile phases to facilitate the fractionation of biological species by a combination of electrophoretic and/or chromatographic mechanisms. A separation system according to one or more embodiments provides mechanical stability of the separating medium, accessibility of the analytes to post-separation characterization techniques (immunodetection, mass spectrometry), ability to fractionate hydrophobic analytes and large molecular complexes, and reduces sample consumption, number of manual manipulations and timelines for performing the actual fractionation.

Planar electrochromatography/thin layer chromatography separations systems and methods are described. The technology described herein is suitable for the separation of a wide range of molecules using a combination of electrically-driven planar chromatography (PEC) and thin-layer chromatography (TLC), such as capillary flow-driven thin layer chromatography, zone refocusing planar chromatography or forced flow planar chromatography, optionally followed by direct detection of analytes using mass spectrometry (MS). The inherent resolving capability of the MS instrument can also be considered an independent separation dimension, as this represents gas phase fractionation according to peptide mass. These three dimensions of separation are substantially orthogonal to one another, providing for improved resolution of the analytes.

In one aspect, a method for separating a sample comprising a plurality of compounds includes loading a sample onto a planar stationary phase, said sample comprising a plurality of compounds to be separated; and while retaining the sample on the planar stationary phase, subjecting the sample to planar electrochromatographic separation in a first direction using a first mobile phase; and subjecting the sample to a chromatographically-based separation in a second direction using a second mobile phase. One or more properties of the first and second mobile phases may be the same or different and may be selected from the group consisting of pH, ionic strength, organic modulator composition and organic content.

In one or more embodiments, the compound is selected from the group consisting of proteins, peptides, amino acids, oligosaccharides, glycans and small drug molecules. The peptide can be a phosphopeptide.

In one or more embodiments, the planar stationary phase comprises an amphiphilic separation medium, and may be a hydrophobic polymer derivatized with ionic groups. For example, the ionic group is selected from one or more of sulfonic acid, sulfopropyl, carboxymethyl, phosphate, diethylaminoethyl, diethylmethylaminoethyl, allylamine and quartenary ammonium residues.

In one or more embodiments, the amphiphilic separation medium is a hydrophobic polymer is selected from the group consisting of polyvinylidine difluoride, polytetrafluoroethylene, poly(methyl methacrylate), polystyrene, polyethylene, polyester, polyurethane, polypropylene, nylon and polychlorotrifluoroethylene.

In one or more embodiments, the planar stationary phase is selected from the group consisting of silica based normal and reverse-phase thin layer chromatography resin derivatized with alkyl groups or aromatic groups.

In one or more embodiments, planar electrochromatographic separation is substantially orthogonal to thin-layer chromatographic separation.

In one or more embodiments, planar electrochromatographic separation comprises applying a voltage across the planar stationary support in contact with a first liquid mobile phase, causing at least one compound of the sample to be displaced in a first direction along the length of the planar stationary phase, wherein the compounds are separated by the partitioning effect between the liquid mobile phase and the solid stationary phase. The separation occurs under electroosmotic forces or under electrokinetic forces.

In one or more embodiments, the chromatographically-based separation comprises thin-layer chromatography, wherein the second mobile phase is advanced by capillary action in a second direction along the planar stationary phase, wherein the compounds are separated by the partitioning effect between the liquid mobile phase and the solid stationary phase.

In one or more embodiments, the chromatographically-based separation is selected from the group consisting zone refocusing planar chromatography and forced flow planar chromatography.

In one or more embodiments, during planar electrochromatographic separation phosphopeptides migrate in a direction opposite that of an unphosphorylated peptide, and phosphopeptides are segregated to a preselected region of the planar stationary phase upon separation.

In one or more embodiments, the planar electrochromatographic separation is done before the chromatographically-based separation, and for example, the first mobile phase is removed before performing said chromatographically-based separation.

In one or more embodiments, the chromatographically-based separation is done before the planar electrochromatographic separation and, for example, the second mobile phase is removed before performing said planar electrochromatographic separation.

The method can further include detecting the separated compounds and, for example, detecting comprises a method selected from the group consisting of fluorescence, mass spectrometry, chemiluminescence, radioactivity, evanescent wave, label-free mass detection, optical absorption and reflection.

In one or more embodiments, the compounds are labeled with a detection agent prior to separation, or after separation.

In one or more embodiments, the detection agent is selected from the group consisting of colored dyes, fluorescent dyes, chemiluminescent dyes, biotinylated labels, radioactive labels, affinity labels, mass tags, and enzymes.

The method can further include subjecting the sample to analysis in a third dimension using mass spectrometry, and for example, mass spectrometry is selected from a group consisting of MALDI-TOF mass spectrometry, ESI-TOF mass spectrometry and inductively-coupled plasma mass spectrometry.

The method can further include mass tagging said compounds for differential analysis of protein expression changes and post-translational modification changes.

In another aspect, a kit for conducting electrochromatography includes a separations plate comprising a planar amphiphilic separation medium for loading a sample comprising one or more compounds; at least one electrode buffer solution suitable for use in planar electrochromatographic separations; and a MALDI-detectable detecting agent.

In another aspect, a kit for conducting electrochromatography includes a separations plate comprising a planar amphiphilic separation medium for separating a sample comprising one or more compounds, and at least one electrode buffer solution suitable for use in planar electrochromatographic separations.

In one or more embodiments, the kit further includes an impermeable barrier to cover said separation medium, wherein said impermeable barrier is glass plate or silicone oil.

In one or more embodiments, the separations plate is a silica TLC plate.

In one or more embodiments, the mobile phase comprises a composition capable to induce migration of phosphopeptides in a direction opposite that of an unphosphorylated peptide, and for example, the mobile phase comprises 1-butanol, pyridine, glacial acetic acid and water.

In another aspect, a method for separating phosphoproteins in a sample includes loading a sample onto a planar stationary phase at a loading origin, said sample comprising a at least one phosphoprotein; and while retaining the sample on the planar stationary phase, subjecting the sample to planar electrochromatographic separation in a selected direction; subjecting the sample to a chromatographically-based separation in a direction other than that of the direction used for planar electrochromatographic separation; and interrogating a preselected region of the planar stationary phase for the presences of phosphopeptides. The preselected region is at or near the loading origin, or anodically located relative to the loading origin.

### BRIEF DESCRIPTION OF DRAWINGS

Various objects, features, and advantages of the present invention can be more fully appreciated with reference to the following detailed description of the invention when considered in connection with the following drawings.

FIG. 1 shows a 2D TLC/TLC separation of P-casein tryptic peptide digest, and demonstrating that 2D TLC/TLC is not an orthogonal separation, as demonstrated by the diagonal distribution of spots in the profile.

FIG. 2 shows a 2D TLE/PEC separation of β-casein tryptic peptide digest; demonstrating that the separation is poor and no anodically migrating peptides are detected.

FIG. 3 shows a separation of β-casein tryptic peptide digest using a silica 60 HPTLC plate with mobile phases of 1-butanol/pyridine/glacial acetic acid/water (50:25:25:900, v/v/v/v, pH 4.7) in the PEC dimension and 2-butanol/acetic acid/pyridine/water (30/6/20/24, v/v/v/v) in the TLC dimension, and demonstrating orthogonal separation.

FIG. 4 illustrates separation of model phosphoprotein tryptic digests (A) ovalbumin, (B) riboflavin binding protein, and (C)HSP 90 using a pH 4.7 solution as the mobile phase in the first dimension and 2-butanol/acetic acid/pyridine/water (30/6/20/24, v/v/v/v) as the mobile phase in the second dimension separation.

FIG. 5 illustrates the migration of (A) model phosphoprotein tryptic digests—(1) α-Casein, (2) ovalbumin, (3) riboflavin binding protein, (4) phosphorylase, (5) human heat shock protein (HSP 90) and (B) model peptides (I.) Insulin receptor peptide, (2.) monophosphorylated kinase domain of insulin receptor, (3.) monophosphorylated kinase domain of insulin receptor-2, (4.) monophosphorylated kinase domain of insulin receptor-4, (5.) triphosphorylated kinase domain of insulin receptor-5, (6.) calcineurin (PP2B) substrate, (7.) monophosphorylated calcineurin (PP2B) substrate, (8.) p60 c-src peptide, (9.) monophosphorylated p60 c-src peptide, and (10.) α-casein tryptic peptide digest, showing that phosphorylated peptides either migrate towards the anode or stay close to the origin while their unphosphorylated counterpart peptides migrate further towards the cathode.

FIG. 6 is a false colour overlap of images representing separations of peptides resulting from the tryptic digest of phosphorylated bovine α-casein S1 (green) and partially dephosphorylated α-casein (magenta), establishing that phosphorylated peptides that move towards the anode in the PEC dimension in the case of phosphorylated α-casein. Matching spots in both images appear in black.

FIG. 7 is a multiplexed detection of peptide spots from β-casein tryptic digest after a ID separation using (A) total protein stain fluorescamine (B) phospho-selective stain Pro-Q Diamond, and (C) MALDI-o-TOF MS based detection of α-casein phosphopeptide directly desorbed from a silica 60 gel-coated plastic-backed TLC plate that has been affixed to a MALDI-TOF MS sample plate.

FIG. 8 illustrates the direct acquisition of mass spectra from a plastic TLC plate using the prOTOF 2000 MALDI-TOF Mass Spectrometer for peptide identification after separation.

### DESCRIPTION OF TECHNOLOGY

The technology described herein includes systems, methods and commercial packages (kits) for separating molecules, such as proteins, peptides, amino acids, oligosaccharides, glycans and small molecules, using a combination of electrically-driven and chromatographic planar separations. More specifically, the technology provides a high resolution two-dimensional, and optionally three-dimensional, separation system that employs a substantially planar solid phase support and combinations of organic and aqueous mobile phases to facilitate the fractionation of biological species by a combination of electrically-driven planar chromatography (PEC) in one dimension and a chromatographic separation in a second dimension, optionally followed by analysis by mass spectrometry (MS) to provide the third dimension. Thin-layer chromatography, zone refocusing planar chromatography and forced flow planar chromatography are useful second dimension fractionation methods. Separation of proteins, peptides, amino acids, oligosaccharides, glycans and small drug molecules are contemplated. A molecule that can be induced to migrate under an electrically-driven separation can be separated using one or more embodiments of the technology. The molecules are typically charged.

Chromatography is a physical separation method in which the components of a mixture are separated by differences in their distribution between two phases, one of which is stationary (stationary phase) while the other (mobile phase) moves through it in a definite direction. The components of the mixture interact with the stationary phase in order to be retained and separated by it. Retention results from a combination of reversible physical interactions that can be characterized in a variety of ways, such as, without limitation, adsorption at a surface, absorption in an immobilized solvent layer, and electrostatic interactions. More than one interaction can contribute simultaneously to a separation mechanism.

Planar electrochromatography (PEC) is a hybrid separations technique in which zone electrophoresis is combined with chromatographic separation. Both chromatographic and electrophoretic processes determine the overall migration rate of the compounds during separation. In contrast, the support phase in conventional electrophoresis does not exhibit any degree of partitioning or hydrophobic interactions.

In electroosmosis-driven planar electrochromatography, the stationary phase can be, for example, an amphiphilic polymeric membrane, amphiphilic thin-layer chromatography plate and the like. The planar stationary phase can be a polymeric membrane, thin-layer chromatography plate, resin-coated MALDI-TOF MS plate and the like. The planar stationary phase surface is characterized by a combination of charge carrying groups (ion exchangers), non-covalent groups, and nonionic groups that facilitate chemical interactions with the analyte, e.g., proteins or peptides.

In a method for the separation of biomolecules using electrochromatographic separation, electro-osmotic flow is generated by application of a voltage across the planar support, in the presence of a miscible organic solvent-aqueous buffer mobile phase. Charged ions accumulate at the electrical double layer of the solid-phase support and move towards the electrode of opposite charge, dragging the liquid mobile phase along with them. Charged biomolecules are separated due to both the partitioning between the liquid phase and the solid phase support and the effects of differential electromigration.

In another method for separation of biomolecules using planar electrochromatography, electrokinetic movement of the analytes is accomplished by application of a voltage across the planar support in the presence of a miscible organic solvent-aqueous buffer mobile phase. The charge of the analyte itself causes movement towards the electrode of opposite charge, without promoting movement of the liquid mobile phase itself. Combinations of both electrokinetic and electroosmotic separation modes are also envisioned.

Thin-layer chromatography is a chromatographic technique for separating and analyzing mixtures of substances using a thin layer of stationary phase attached to a substrate and using the passage of liquid (mobile phase) in one direction along the stationary phase as a means for separation. The stationary phase is immobilized on the support, for example, by using a binder to impart the desired mechanical strength and stability to the layer. The mixture is applied to the layer as spots or bands near the bottom edge of the plate. The separation is achieved by contacting the bottom edge of the plate below the line of samples with the mobile phase, which proceeds to ascend the layer by capillary action. Other method of thin-layer chromatography include zone refocusing planar, wherein natural zone broadening is counteracted by, for example, employment of multiple development steps with different mobile phases or forced flow planar chromatography, wherein an external force, such as pressure, is used to move the mobile phase through the chromatography layer, typically at a constant velocity corresponding to the optimum mobile phase velocity, but in any case at any other selected velocity. In one or more embodiments, the combination of PEC- and TLC-based fractionation provides for a separation of peptides on the basis of charge-to-mass ratio and hydrophobicity.

In one or more embodiments, the different dimensions of separation are substantially orthogonal, that is, the separations are statistically independent. Because the different separations methods separate using different physicochemical properties of the molecules, the separations power of the PEC/TLC separations method is very powerful. Although individually the resolving power of the first two fractionation approaches is modest, together with MS, the separation efficiency of the system is impressive. For example, assume that the PEC- and TLC-based separations are each only capable of delineating 25 features, while the MS is capable of resolving 10,000 features. For this example, the composite potential resolving power of the multidimensional peptide separation system is 6,250,000 species.

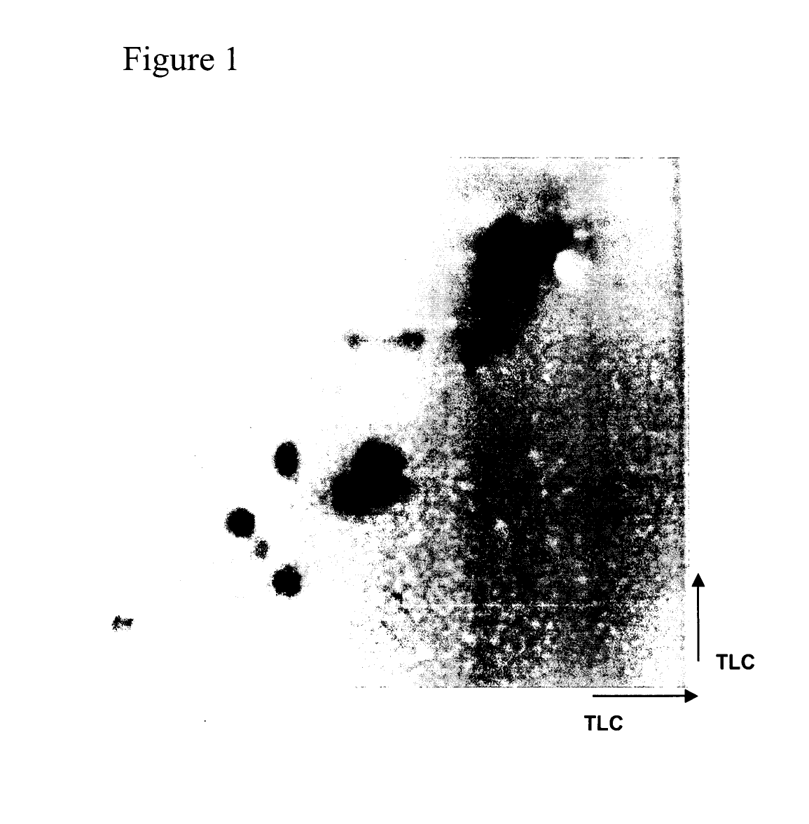

Orthogonal 2D separation using a TLC-based approach (TLC/TLC) has not been observed. FIG. 1 depicts a typical 2D separation of a β-casein tryptic peptide digest using and acid buffer (2-butanol/acetic acid/pyridine/water (30/6/20/24, v/v/v/v)) and a basic buffer (2-butanol/ammonia (25%)/pyridine/water (39/10/2/26, v/v/v/v)) serving as the mobile phases. It is apparent from the profile that the two TLC separations are not completely orthogonal, that is the migration of the peptides in the first dimension is somewhat correlated with their migration in the second dimension.

An analogous separation of a β-casein tryptic peptide digest performed by 2D PEC/PEC also was not successful. Pre-wetting the plate before the PEC run in the second dimension was challenging as the peptide spots separated in the first dimension easily diffused away resulting in dispersed instead of compact spots.

Two dimensional peptide separations on cellulose TLC plates using a combination of thin-layer electrokinetic/electroosmosis (TLE) fractionation in the first dimension followed by thin-layer chromatography in the second dimension (TLE/TLC) also have been attempted, but have not provided sensitive or efficient separations. TLE is different from PEC in that there is no significant interaction of the analyte with the chromatographic matrix in the former technique. This method is widely used for the analysis of ³²P-labeled phosphopeptides, and separations are typically performed on cellulose, not silica TLC plates. See, van der Geer P, Hunter T., “Phosphopeptide mapping and phosphoamino acid analysis by electrophoresis and chromatography on thin-layer cellulose plates,” Electrophoresis 1994 March-April; 15(3-4):544-554; and Stephens R. E., “Fluorescent thin-layer peptide mapping for protein identification and comparison in the subnanomole range,” Anal Biochem. 1978 January; 84(1): 116-26.).

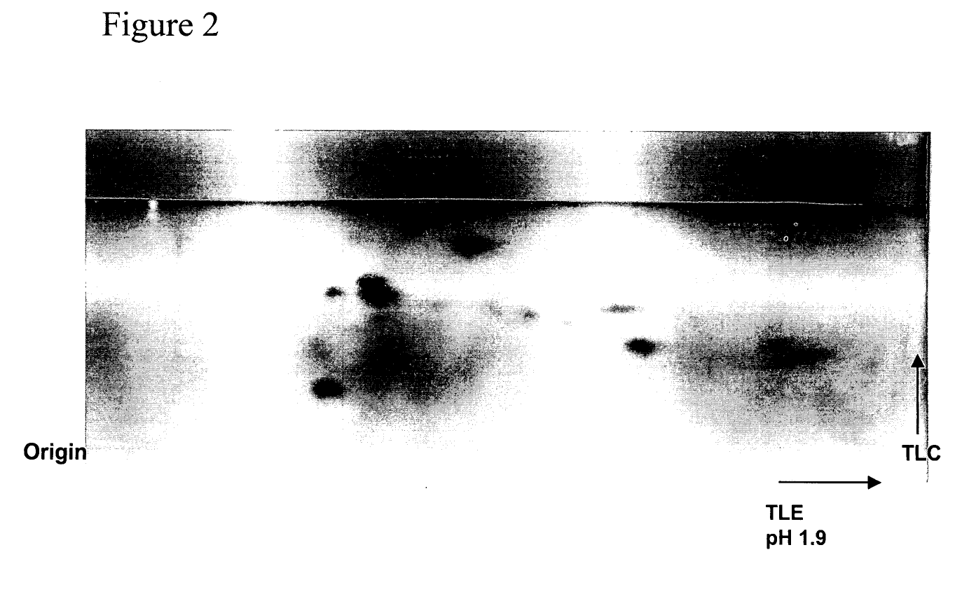

FIG. 2 shows a typical 2D separation of a α-casein tryptic peptide digest using a combination of TLE and TLC. In this separation, 88% formic acid/glacial acetic acid/water, pH 1.9 (50:156:1794 v/v/v) was used as the mobile phase for the TLE dimension and n-butanol/pyridine/glacial acetic acid/water (75/50/15/60 v/v/v/v) was employed for the TLC dimension. The separation results in a relatively low number of detected spots. Also, no anodically migrating peptides were detected.

In contrast, the technology provides a completely orthogonal separation of peptides. This combination of PEC- and TLC-based fractionation provides for a separation of peptides on the basis of charge-to-mass ratio and hydrophobicity, respectively, two attributes recognized to change upon covalent attachment of a phosphate group. The separation method is particularly well-suited for the separation of acidic peptides, such as phosphopeptides, which are typically too hydrophilic to separate efficiently in chromatographic separations. Such molecules can demonstrate anodic migration in a direction opposite of bulk peptide migration.

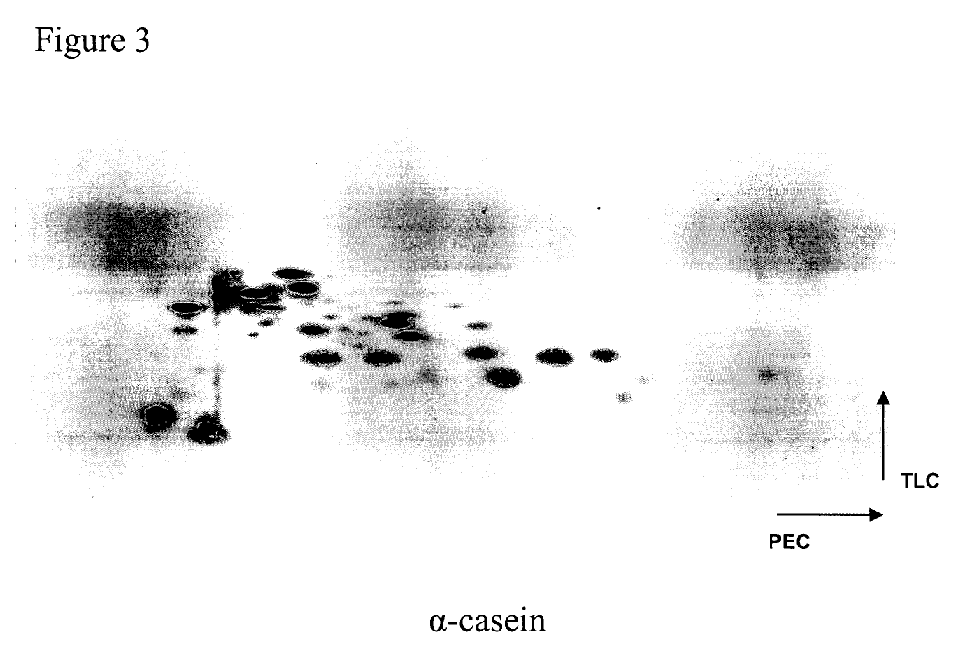

FIG. 3 shows a 2D separation approach for α-casein digest, performed on silica gel 60 glass-backed HPTLC plates using a combination of PEC, with n-butanol/pyridine/glacial acetic acid/water (50/25/25/900, v/v/v/v) as the mobile phase and TLC, with 2-butanol/acetic acid/pyridine/water (30/6/20/24, v/v/v/v) as the mobile phase. The phosphorylated peptides in the digest or synthetic standards either migrated in the opposite direction of the bulk peptides or stayed close to the origin during PEC. The putative phosphopeptide spots were too hydrophilic to migrate substantially during the subsequent TLC separation, resulting in them only being displaced primarily in an anodic direction relative to the origin (just left of the origin in the Figure). Performing analogous separations with a cellulose solid phase support resulted in substandard profiles. Furthermore, coupling of cellulose-based TLC matrices to MALDI-TOF MS is complicated by the presence of contaminating polymers derived from the resin.

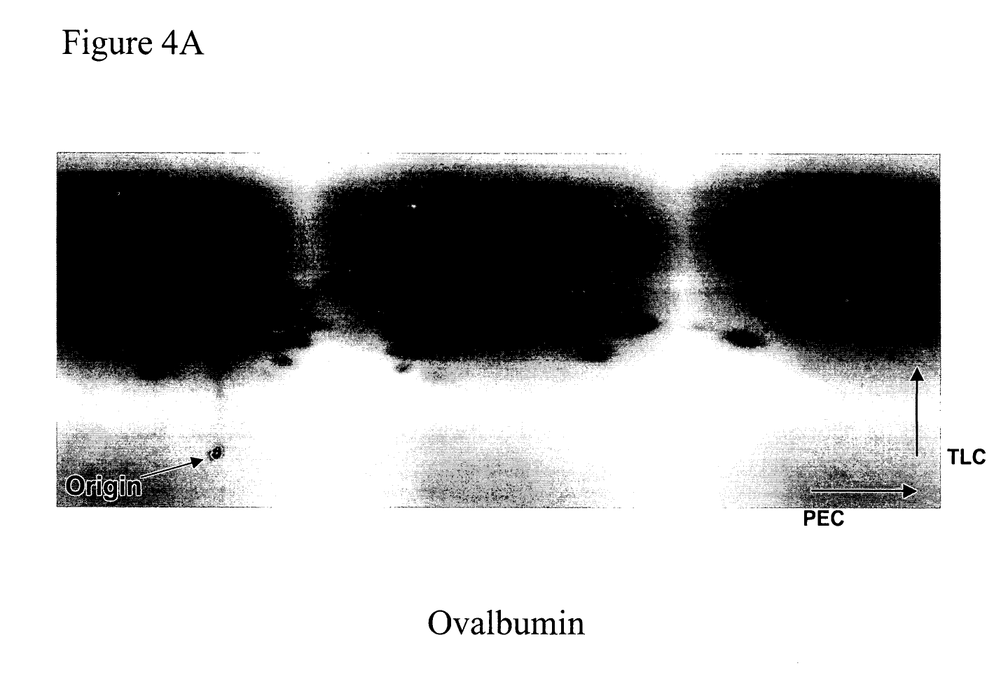

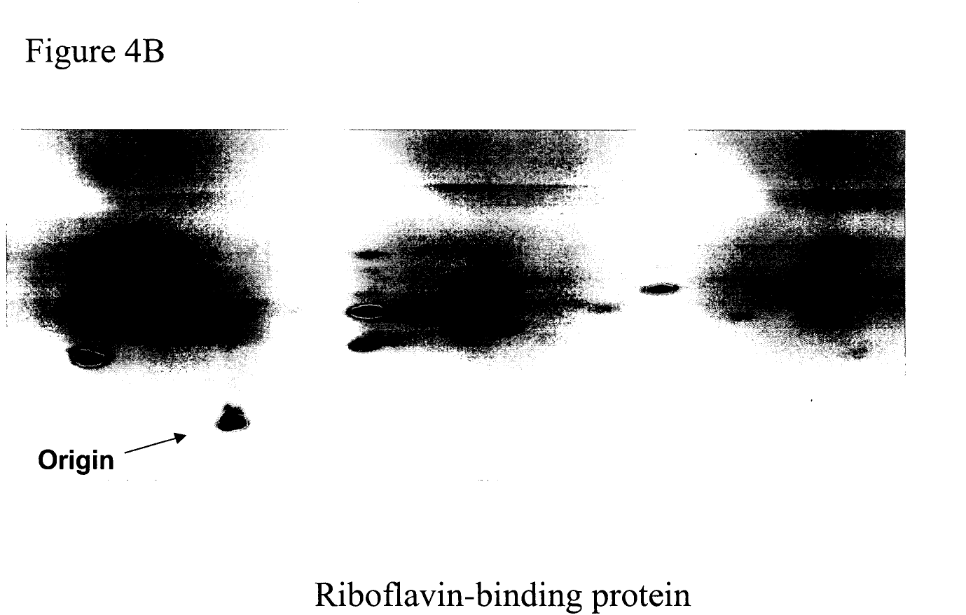

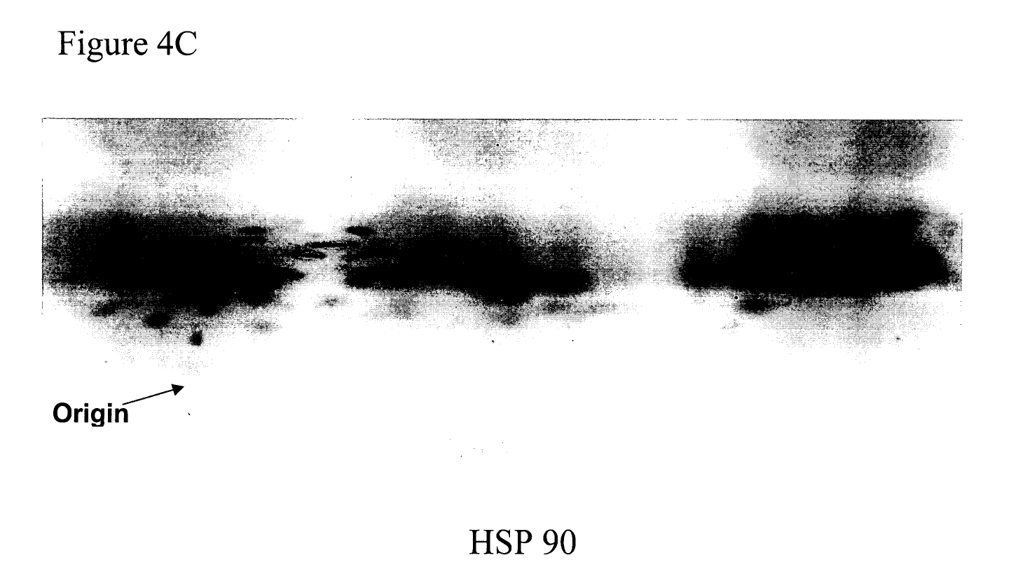

Using this two dimensional PEC/TLC method, separation of various other model phosphoprotein tryptic digests was performed. FIG. 4 shows the separation for ovalbumin (FIG. 4A), riboflavin-binding protein (FIG. 4B), and HSP 90 (FIG. 4C). In all these 2D separations, a minority of peptide spots moved towards the anode and to some extent, these spots migrated in the second dimension as well.

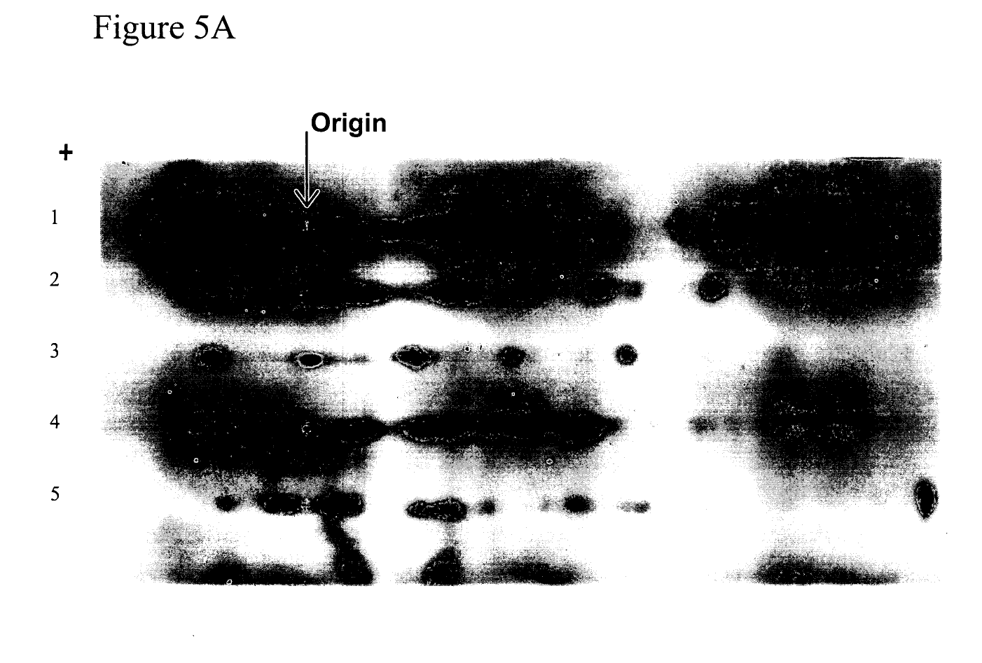

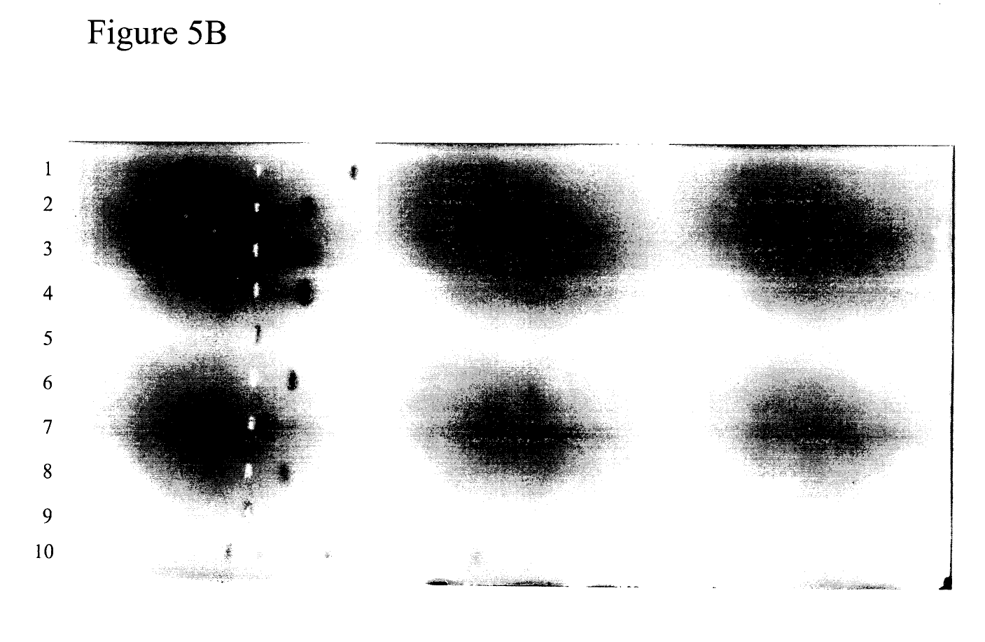

In order to verify that prominent anodically migrating peptides observed in PEC are phosphorylated, tryptic digests of model phosphoproteins and commercially available synthetic phosphopeptide standards were spotted on the HPTLC plates and separated using PEC. FIG. 5A illustrates the migration and separation of model phosphoprotein tryptic digests (1) α-casein, (2) ovalbumin, (3) riboflavin binding protein, (4) phosphorylase a, and (5) human heat shock protein (HSP 90) during PEC on a silica gel 60 glass-backed HPTLC plate. In each of these lanes, a subset of peptide spots migrated towards the anode. The PEC-based migration phenomenon was studied further using a battery of model peptides and phosphopeptides, as well as with a β-casein tryptic peptide digest. Insulin receptor peptide, monophosphorylated kinase domain of insulin receptor, monophosphorylated kinase domain of insulin receptor-2, monophosphorylated kinase domain of insulin receptor-4, triphosphorylated kinase domain of insulin receptor-5, calcineurin (PP2B) substrate monophosphorylated calcineurin (PP2B) substrate, p60 c-src peptide and monophosphorylated p60 c-src peptide were employed in the experiments. FIG. 5B shows the PEC separation of these model synthetic peptides and their phosphopeptide variants clearly indicating that the phosphorylated peptides remained closer to the spotted origin. However, the model phosphopeptides were not displaced anodically, as observed with the tryptic digest peptides. At the same time, both phosphorylated peptides of the tryptic digest of α-casein moved in the opposite direction of the bulk peptides, towards the anode. In one or more embodiments, a decrease in pI observed upon phosphorylation correlates with a decreased cathode migration upon PEC. In one or more embodiments, larger peptides with lower pI tend to display a decreased cathode migration and in some instances anodic migration in PEC. In one or more embodiments, the phosphopeptides migrating anodically contain a high percentage of acidic amino acid residues, such as aspartic and glutamic acid residues (for example, the phosphopeptides of β-casein).

The technology described herein can provide, for example, the ability to fractionate larger amounts of sample relative to previously described techniques, ability to re-analyze fractions and the ability to use commercially available, off-the-shelf equipment that requires minimal, if any instrumental modification; ability to perform simultaneous parallel separations; static detection free of time constraints; and ability to observe substantially all, or most sample components in the chromatogram. If desired, the materials used for performing methods described herein can include disposable components, such as a disposable stationary phase; as well as information storage components, such as a storage device for chromatographic information.

The technology described herein can involve providing a sample containing one or more molecules e.g., charged molecules, e.g., biomolecules, loading the sample on a planar stationary phase, contacting the stationary phase with a first liquid mobile phase, providing first and second electrodes in electrical contact with opposing edges of the stationary phase; and creating an electrical field between the first electrode and the second electrode so as to cause the first liquid mobile phase and/or the sample to advance across the length of the stationary phase, whereby one or more biomolecules are separated. The biomolecule can be selected from, for example, proteins, peptides, amino acids, oligosaccharides, glycans and small drug molecules. The pH, ionic strength and water/organic content of the mobile phase can be selected to promote electrokinetic and/or electroosmosis-driven separation. The liquid mobile phase can be, for example, an aqueous mixture containing a water miscible organic liquid. The liquid mobile phase can be selected from, for example, methanol-aqueous buffer; acetonitrile-aqueous buffer; ethanol-aqueous buffer; isopropyl alcohol-aqueous buffer; butanol-aqueous buffer; isobutyl alcohol-aqueous buffer; carbonate-aqueous buffer; furfuryl alcohol-aqueous buffer; and mixtures thereof. The liquid mobile phase can have a composition that induces migration of acidic peptides, e.g., negatively charged molecules such as phosphorylated peptides, in an opposite direction as the majority of the bulk, e.g., unphosphorylated, peptides upon application of an electric field. An example of such a liquid mobile phase is 1-butanol/pyridine/glacial acetic acid/water (50:25:25:900, v/v/v/v, pH 4.7).

The technology described herein involves applying a second separation that is chromatographically-based, so as to cause a second liquid mobile phase to be advanced across the length of the stationary phase in a second direction, whereby one or more biomolecules are separated. The pH, ionic strength and water/organic content of the mobile phase can be selected to promote chromatographic separation in this second direction. Examples of suitable mobile phase include 2-butanol/ammonia (25%)/pyridine/water (39/10/2/26, v/v/v/v) and 2-butanol/acetic acid/pyridine/water (30/6/20/24, v/v/v/v).

In one or more embodiments, the chromatographic separation can be performed, for example, as the first dimension separation and the electrically-driven separation as the second dimension separation. In one or more embodiments, the planar electrochromatographic separation and the thin-layer chromatographic separation can be performed in any order.

In one or more embodiments, the chromatographic mobile phase can be selected to impede or promote migration of phosphopeptides relative to the bulk peptides in the sample, if desired. The first and second mobile phases generally have different pH values. As one example, the pH of the first mobile phase is acidic and the pH of the second mobile phase is basic; and in other aspects, the pH of the first mobile phase is basic and the pH of the second mobile phase is acidic. As another example, the pH value of the two mobile phases are similar, that is both acidic, both neutral, or both basic. The first and second mobile phase can also have different organic solvent content. As one example, the first liquid mobile phase can have a higher organic solvent concentration than the second liquid mobile phase. As another example, the first liquid mobile phase can have a lower organic solvent concentration than the second liquid mobile phase. The first and second liquid mobile phases can have different ionic strengths.

Upon completion of separation in one direction, e.g., the first dimension separation, the solid phase can be rinsed, or the mobile phase allowed to evaporate away prior to a second organic solvent-aqueous buffer mobile phase being introduced at one edge of the planar solid phase support and the analyte is then fractionated in a direction that differs from the original direction of separation (e.g., the second dimension separation) by a chromatographic mechanism (e.g. TLC, zone refocusing planar chromatography or forced flow planar chromatography). Typically, the second direction is perpendicular to the first direction. One dimension can be separated by the partitioning effects between the liquid phase and solid support and the other thru effects of electromigration. By adjusting the pH, ionic strength and organic solvent concentration, and whether or not an electric field is applied, electrophoretic/electrooosmotic separation in one dimension is obtained and separation in second dimension is obtained chromatographically.

The technology as described in one or more embodiments herein is useful for the analysis of phosphomolecules, as well as phosphorylation sites in proteins. Using these systems and methods according to one or more embodiments, phosphorylated peptides can be induced to migrate in the opposite direction as the bulk of the other peptides in the first dimension, based upon charge properties and are further distinguished from adventitial peptides based upon hydrophilicity in the second dimension. This permits a restricted region of the plate to be interrogated for the presence of phosphopeptides by mass. This phosphopeptide mapping approach can allow routine identification of phosphopeptides without the use of radioisotopes or surrogate dyes.

Separating tryptic peptides according to charge using strong-cation exchange chromatography at a pH value of 2.7 has been described, for example, in US 2005/0164324 A1. It is known that at pH 2.7, only lysine, arginine, histidine, and the amino terminus of a peptide are charged, and that trypsin proteolysis produces peptides with a C-terminal lysine or arginine residue. Thus, most tryptic peptides carry a net solution charge state of plus two. Because a phosphate group maintains a negative charge at acidic pH values, the net charge state of a phosphopeptide is generally only plus one. However, peptides having other post-translational modifications, such as acetylated peptides, co-elute with the phosphopeptides using the prior art method. Using the technology described herein, peptides having such post-translational modifications can be separated away from phosphopeptides based upon the hydrophobicity-based TLC fractionation, if desired.

The technology described herein can involve using an amphiphilic stationary phase. An amphiphilic stationary phase refers to a solid-support stationary phase exhibiting both non-polar and polar interactions with the analyte, e.g., proteins, glycans or peptides and the like. An amphiphilic stationary phase can include regions, phases or domains that are nonionic and/or hydrophobic in nature as well as regions, phases or domains that are highly polar and in some cases ionic. The ionic regions can be positively or negatively charged. Hydrophobic groups favor the interaction and retention of the protein during separation, while the ionic groups promote the formation of the charged double layer used in electrokinetic separation. In one aspect, the amphiphilic stationary phase for protein fractionation has a combination of charge carrying groups (ion exchangers), non-covalent groups, and nonionic groups that facilitate chemical interactions with the analytes. In another aspect, the amphiphilic stationary phase is predominantly hydrophobic, but partially ionic in character. Examples of amphiphilic stationary phases that can be used for protein separation can include hydrophobic planar support derivatized with sulfonic acid, sulfopropyl, carboxymethyl, phosphate, diethylaminoethyl, diethylmethylaminoethy, allylamine or quartenary ammonium residues or the like. Hydrophobic planar supports derivatized with sulfonic acid, sulfopropyl, carboxymethyl, or phosphate residues enable cathodic electroosmotic flow, while hydrophobic planar supports derivatized with diethylaminoethyl, diethylmethylaminoethy, allylamine or quartenary ammonium residues enable anodic electroosmotic flow. Membranes, particulate thin-layer chromatography substrates, large pore mesoporous substrates, grafted gigaporous substrates, gel-filled gigaporous substrates, nonporous reversed phase packing material and polymeric monoliths can also be used in the technology described herein.

A planar stationary phase useful in the technology described herein can include a silica, alumina or titania based thin layer chromatography resin, either underivatized or derivatized with alkyl groups, aromatic groups, or cyanoalkyl groups. The planar stationary phase can include silica, alumina or titania-particles derivatized with alkyl, aromatic or cyanoalkyl groups. The planar stationary phase can include pores of about 30 nanometers to about 100 nanometers in diameter. The planar stationary phase can be made up of particles having a diameter of about 3 microns to about 50 microns. An amphiphilic planar stationary phase useful in the technology described herein can include a hydrophobic polymer derivatized with ionic groups. The ionic group can be selected from, for example, one or more of sulfonic acid, sulfopropyl, carboxymethyl, phosphate, diethylaminoethyl, diethylmethylaminoethyl, allylamine and quartenary ammonium residues. The hydrophobic polymer can be selected from, for example, polyvinylidine difluoride, polytetrafluoroethylene, poly(methyl methacrylate), polystyrene, polyethylene, polyester, polyurethane, polypropylene, nylon and polychlorotrifluoroethylene. The derivatized hydrophobic polymer can be particulate.

The technology described herein can be carried out using membrane-based electrochromatography. Membranes include polymeric sheets, optionally derivatized to provide the amphiphilic character of the planar stationary phase. Exemplary hydrophobic membranes for membrane-based electrochromatography of proteins and peptides include Perfluorosulfonic Nafion® 117 membrane (Dupont Corporation), partially sulfonated PVDF membrane, sulfonated polytetrafluoroethylene grafted with polystyrene, polychlorotrifluoroethylene grafted with polystyrene, or the like. Sulfonation of polyvinylidene difluoride (PVDF) can be achieved by incubation with sulfuric acid at a moderately high temperature. The degree of sulfonation can be systematically varied, where ion exchange capability of 0.25 meq/g is considered as moderate sulfonation. In these membranes separation depends upon the electrostatic interaction of proteins with sulfonated residues in combination with hydrophobic interactions with aromatic residues in the substrate. At pH in the range from about pH 2.0 to about pH 11.0, the protonated primary amine groups on the proteins will interact with sulfonated residues on the membrane. This interaction is diminished at pH greater than about pH 11.0. Sulfonate residues will be protonated at a pH less than about pH 2.0 and will lead to a decline in the electroosmosis driving force of the separation.

PVDF membranes can be useful in the methods described herein. For example, these membranes can be derivatized with cationic functional groups in order to generate an amphiphilic membrane (e.g., Immobilon-CD protein sequencing membrane (Millipore Corporation)). PVDF membrane can be etched with 0.5 M alcoholic KOH and subsequently reacted with polyallylamine under alkaline conditions, if desired. As another example, PVDF membranes can be derivatized with diethylaminoethyl or quartenary ammonium residues. The membrane also can be unsupported, supported or semi-supported. For example, the membrane can be held between two rigid or semi-rigid holders in the form of frames with large openings in the center. The membrane can also be rigidly supported on a solid support, for example, a glass plate. Membranes can be substantially non-porous. In such instances, the mobile phase moves over the surface of the membrane. In other aspects, the membrane can be porous, in which case the mobile phase moves through the pores and/or channels of the membrane. Separation is thought to occur by preferential interactions of the proteins with the hydrophobic surfaces or the interstitial surfaces of the membrane.

Further planar stationary phases useful for separation of proteins include silica thin-layer chromatography plates underivatized or derivatized with alkyl groups (e.g. C₃-C₁₈ surface chemistry), aromatic phenyl residues, cyanopropyl residues or the like. In these instances, the silanol groups provide the ion exchange qualities of the amphiphilic support and can be deprotonated at a pH of 8, leading to electroosmosis and thereby providing the ion exchange qualities of the amphiphilic support. At pH below pH 3, there will be a reduction or elimination in electroosmosis. In some aspects, both hydrophobic groups, e.g., alkyl, and charged groups, e.g., sulfonic acid, can be attached to the same silica particle. As a further example, a stationary phase support for the separation of peptides and proteins according to one or more embodiments described herein can include a gamma-glycidoxypropyltrimethoxysilane sublayer attached to the silica support of a thin-layer chromatography plate. A sulfonated layer is then covalently affixed between the sublayer and an octadecyl top layer. For separation of proteins in the 10 and 100 kDa range using a silica-based stationary phase, it is expected that derivitization with C8 and C4 groups, respectively, can be used. Phenyl functionalities are slightly less hydrophobic than C4 functionalities and can be advantageous for the separation of certain polypeptides.

A planar stationary phase used in the methods described herein can include pores or connected pathways of a dimension that permits unimpeded migration of the proteins. For particulate stationary phases, such as silica thin-layer chromatography plates or particulate-based polymer membranes, the stationary phase can consist of particles that form pores of about 30-100 nanometers in diameter, although for some smaller peptides with molecular weights of 2,000 daltons or less, 10 nanometers pores can be useful. Typical absorbants commercially available for thin-layer chromatography are made of particles that form pores sizes of only 1-6 nm, which precludes effective use for protein separations. The particles can have a diameter of about 3-50 microns, with the smaller diameter particles typically producing higher resolution protein separations. For higher protein loads, large particle absorbents are generally used, such as for preparative scale isolation of proteins. The size distribution of the particles is generally relatively narrow and particles are typically spherical. The base material of the particles can be silica, synthetic polymers, such as polystyrene-divinylbenzene (or any of the above mentioned hydrophobic polymers) and the like. Example 6 describes fabrication of a titania-based support with large pores useful for protein separations.

For further detail regarding apparatus, systems and methods for performing planar electrochromatographic separations, see co-pending and commonly owned United States Published application Ser. No. 11/084,501, entitled “Separations Platform Based Upon Electroosmosis-Driven Planar Chromatography” the contents of which are incorporated by reference in its entirety.

A liquid mobile phase useful in the methods described herein can include an organic phase and an aqueous phase. Exemplary mobile phases include methanol-aqueous buffer, acetonitrile-aqueous buffer, ethanol-aqueous buffer, isopropyl alcohol-aqueous buffer, butanol-aqueous buffer, isobutyl alcohol-aqueous buffer, propylene carbonate-aqueous buffer, furfuryl alcohol-aqueous buffer systems or the like. The basic principles of electrochromatography and thin layer chromatography provide the foundation for systematic selection of stationary phase supports, mobile phase buffers and operating conditions. Mobile phases rich in organic modulators generally exhibit relatively little chromatographic retention and in mobile phases low in organic modulator, chromatographic retention will dominate the separation process.

For the methods described herein, the concentrations of organic modulators in liquid mobile phases are in the range of about 0% to about 60%. In another aspect, the ionic strength of liquid mobile phases can be from about 2 mM to about 150 mM. Exemplary liquid mobile phase formulations include 20 mM ammonium acetate, pH 4.4,20% acetonitrile; 2.5 mM ammonium acetate, pH 9.4, 50% acetonitrile; 25 mM Tris-HCl, pH 8.0/acetonitrile (40/60 mix); 10-25 mM sodium acetate, pH 4.5, 55% acetonitrile; 60 mM sodium phosphate, pH2.5/30% acetonitrile; 5 mM borate, pH 10.0, 50% acetonitrile; 5-20 mM sodium phosphate, pH 2.5, 35-65% acetonitrile; mM potassium phosphate, pH 3.0, 60% acetonitrile and 10 mM sodium tetraborate, 30% acetonitrile, 0.1% trifluoroacetic acid; 20% methanol, 80% 10 mM MES, pH 6.5, 5 mM sodium dodecyl sulfate; 20% methanol, 80% 10 mM MES, pH 6.5, 5 mM sodium phosphate, pH 7.0/methanol (4:1, v/v); 4 mM Tris, 47 mM glycine, pH 8.1; 20 mM sodium phosphate, pH 6.0, 150 mM NaCl; 20 mM Tris-HCl, pH 7.0, 150 mM NaCl; 5 mM sodium borate, pH 10.0; or the like.

For the methods described herein, different cathode and anode buffers can be used as a discontinuous buffer system for the separation of proteins. In certain of these aspects, the amphiphilic stationary phase can be incubated in a buffer that is compositionally different from either electrode buffer. Additives, such as carrier ampholytes can be included in the buffer in which the stationary phase is incubated. In other aspects, the composition of the mobile phase can be altered temporally to provide a composition gradient that facilitates separation of proteins.

In two-dimensional separation of proteins on an amphiphilic stationary phase, using a method according to one or more aspects of the technology described herein, protein sample can be applied on the center of the membrane (dry or pre-wetted with mobile phase) and the planar stationary phase is then incubated in a mobile phase. Once the proteins are electrophoretically separated in one direction, the planar stationary phase is evaporated away and incubated in a second mobile phase, and then chromatographically separated in a direction perpendicular to the first direction. In one aspect in accordance with the technology described herein, liquid mobile phases can be adjusted to different pH values, concentrations of organic solvent, and/or ionic strengths to facilitate 2D separations of proteins or peptides on the amphiphilic substrate. For example, one mobile phase will have acidic pH (ca. pH 4.5) and the other basic pH (ca pH 8.5). The pH of the buffers will affect the total charge of the individual protein species and thus influence their electrokinetic migration. Changes to the concentration of organic solvent in liquid mobile phase impacts the strength of interaction of the proteins with the hydrophobic component of the stationary phase. Finally, the ionic strength of the buffer changes as the separation properties of the proteins in the two dimensions. By manipulating pH, ionic strength and organic solvent concentration, separation in one dimension will occur electrophoretically and separation in the other dimension will occur chromatographically.

Protein samples for use in a method described herein can be prepared, for example, by dissolving the proteins in the mobile phase or a weaker solvent of lower ionic strength. A biological buffer, such as Good's buffers, is an example of a solution for preparing protein samples. These biological buffers produce lower currents than inorganic salts, thereby allowing the use of higher sample concentrations and higher field strengths. Exemplary Good's buffers include N-(2-Acetamido)-2-aminoethanesulfonic acid (ACES), N-(2-Acetamido)iminodiacetic acid (ADA), N,N-Bis(2-hydroxyethyl)-2-aminoethanesulfonic acid (BES), N,N-Bis(2-hydroxyethyl)glycine (BICINE), Bis(2-hydroxyethyl)iminotris(hydroxylmethyl)methane(BIS-TRIS), N-Cyclohexyl-3-aminopropanesulfonic acid (CAPS), N-Cyclohexyl-2-hydroxy-3-aminopropanesulfonic acid (CAPSO), N-Cyclohexyl-2-aminoethanesulfonic acid (CHES), 3-[N,N-Bis(hydroxyethyl)amino]-2-hydroxypropanesulfonic acid (DIPSO), 3-[4-(2-Hydroxyethyl)-1-piperazinyl]propanesulfonic acid (EPPS), 2-[4-(2-Hydroxyethyl)-1-piperazinyl]ethanesulfonic acid (HEPES), 2-Hydroxy-3-[4-(2-hydroxyethyl)-1-piperazinyl]-propanesulfonic acid, monohydrate (HEPPSO), 2-Morpholinoethanesulfonic acid, monohydrate (MES), 3-Morpholinopropanesulfonic acid (MOPS), 2-Hydroxy-3-morpholinopropanesulfonic acid (MOPSO), piperazine-1,4-bis(2-ethanesulfonic acid) (PIPES), piperazine-1,4-bis(2-ethanesulfonic acid), sesquisodium salt (PIPES, sesquisodium salt), piperazine-1,4-bis(2-hydroxy-3-propanesulfonic acid), dehydrate (POPSO), N-Tris(hydroxymethyl)methyl-3-aminopropanesulfonic acid (TAPS), N-Tris(hydroxymethyl)methyl-2-hydroxy-3-aminopropanesulfonic acid (TAPSO), Tris-(hydroxymethyl)aminomethane (TRIS), N-Tris(hydroxymethyl)methyl-2-aminoethanesulfonic acid (TES), and N-[Tris(hydroxymethyl)methyl]glycine (TRICINE). If salts are used to facilitate extraction and isolation of the protein specimen, desalting of protein samples can be performed using reverse phase resins by organic solvent-based protein precipitation or by sample dialysis prior to sample fractionation by planar electrochromatography.

Protein samples for use in a method described herein can also be prepared by dissolving the proteins in HPLC solvent systems thereby avoiding the use of detergents, chaotropes and strong organic acids for protein dissolution. HPLC solvent systems include buffered solutions containing organic solvents, such as methanol or acetonitrile, can be employed to prepare the biological specimens. For example, 60% methanol or acetonitrile, 40% water containing 0.1% formic acid or 60% methanol or acetonitrile, 40% 50 mM ammonium carbonate, pH 8.0 are suitable sample solubilization buffers. In one aspect, final protein concentration in the solubilization buffer is from about 0.05 mg/ml to about 5 mg/ml. In another aspect, final protein concentration in the solubilization buffer is from about 0.4 mg/ml to about 0.6 mg/ml. Extraction and solubilization of proteins can be facilitated by intermittent vortexing and sonication. Surfactants are well known to suppress peptide ionization in mass spectrometry and also to interfere with chromatographic separations, particularly with reversed-phase liquid chromatography. Buffered solutions containing organic solvents are more compatible with liquid chromatography and mass spectrometry and thus facilitate characterization of the proteins after planar electrochromatography. Another important advantage of the buffered organic solvent extraction procedure is that it facilitates solubilization, separation and identification of integral membrane proteins, including proteins containing transmembrane-spanning helices.

Planar electrochromatographic separation of peptides and proteins is performed by directly applying an electric field across the membrane or TLC plate. In one aspect, the planar surface can be interfaced with the electrical system through the use of wicks, also referred to as buffer strips. A wick is a solid or semisolid medium used to establish uniform electrical paths between the planar solid phase and the electrodes of a horizontal electrophoresis apparatus. For example, a wick can be composed of cellulose-based filter paper, Rayon fiber, buffer-impregnated agarose gel, moistened paper towel, or the like.

Application of an electric field in electrochromatographic systems can result in Joule heating which in turn can to lead to evaporation of liquid mobile phase from the membrane or plate surface. The evaporation of the mobile phase can result in decreased current, drying of the surface, and subsequent degradation in the quality of the separation. In one aspect in accordance with the technology described herein, the planar stationary phase is covered with a glass plate, silicone oil or other impermeable barrier to reduce the evaporation of the mobile phase as a result of Joule heating. Further, flow of the mobile phase across the membrane or plate can be impeded in the forward direction, causing the electroosmotic flow to drive the liquid mobile phase to the surface of the membrane or plate. This can result in poor resolution separations and arcing of the electrophoretic device. Adjusting mobile phase pH or ionic strength will aid in optimizing conditions for the electrically driven separation. In one aspect, operating current for protein or peptide separations is from about 10 μA to about 500 mA and the electric field strength applied to the separation is from about 50 volts/cm to about 900 volts/cm. In another aspect, the electric field strength applied to the separation is from 200 volts/cm to about 600 volts/cm. In certain aspects of the technology described herein, separations of proteins can be performed using constant voltage, constant current or constant power mode, the latter resulting in constant amount of Joule heating in the system. See, United States Published application Ser. No. 11/084,501, entitled “Separations Platform Based Upon Electroosmosis-Driven Planar Chromatography” the contents of which are incorporated by reference in its entirety.

The methods described herein also can involve detecting the separated molecules, for example, by detecting fluorescence, mass spectrometry, chemiluminescence, radioactivity, evanescent wave, label-free mass detection, optical absorption and reflection. The biomolecules can be labeled with a detection agent prior to or after separation. The detection agent can be selected from, for example, colored dyes, fluorescent dyes, chemiluminescent dyes, biotinylated labels, radioactive labels, affinity labels, mass tags, and enzymes. The separations method can include mass tagging the biomolecules for differential analysis of protein expression changes and post-translational modification changes.

Proteins, peptides and glycans can be detected after using a variety of detection modalities well known to those skilled in the art. Exemplary strategies employed for general protein detection include organic dye staining, silver staining, radio-labeling, fluorescent staining (pre-labeling, post-staining), chemiluminescent staining, mass spectrometry-based approaches, negative-staining approaches, contact detection methods, direct measurement of the inherent fluorescence of proteins, evanescent wave, label-free mass detection, optical absorption and reflection, or the like. In negative-staining approaches, the proteins remain unlabeled, but unoccupied sites on the planar surface are stained. In contact detection methods, another membrane or filter paper that has been imbibed with a substrate is placed in contact with the planar surface and protein species resident on the planar stationary phase interact with the substrate molecules to generate a product. In direct measurement of the inherent fluorescence of proteins, solid-phase supports of low inherent fluorescence are used. Exemplary detection methods suitable for revealing protein post-translational modifications include methods for the detection of glycoproteins, phosphoproteins, proteolytic modifications, S-nitrosylation, arginine methylation and ADP-ribosylation. Exemplary methods for the detection of a range of reporter enzymes and epitope tags include methods for visualizing β-glucuronidase, (β-galactosidase, oligohistidine tags, and green fluorescent protein. For optimal performance of these detection technologies, it will be necessary to use solid-phase supports of low inherent fluorescence.

Protein samples that have undergone separation according to one or more embodiments described herein generally appear as discrete spots on the plate that are accessible to staining or immunolabeling as well as to analysis by various detection methods. Exemplary detection methods include mass spectrometry, Edman-based protein sequencing, or other micro-characterization techniques. In one aspect, proteins bound to the surface of the membrane can be labeled by reagents, such as, antibodies, peptide antibody mimetics, oligonucleotide aptamers, quantum dots, Luminex beads or the like. Chemiluminescence-based detection of proteins on planar surfaces can be used prior to or after fractionation by. In addition, proteins can be biotinylated and then detected using horseradish peroxidase-conjugated streptavidin and the Western Lightning Chemiluminescence commercial package (PerkinElmer). Further, proteins can be fluorescently stained or labeled and the fluorescent dye subsequently chemically described by nonenzymatic means, such as the bis(2,4,6-trichlorophenyl)oxalate (TCPO)—H₂O₂ reaction.

Separations of protein or peptide, using the technology described herein, can be achieved in a short duration. As an example, samples are spotted on a planar stationary phase, subjected to first dimension separation, solvent evaporated away and subjected to second dimension chromatographic separation thereby providing access to the proteins and peptides on the surface of the stationary phase for detection. SYPRO Ruby protein blot stain (Molecular Probes) is an exemplary dye capable of detecting proteins on a surface within about 15 minutes. Additionally, the planar support itself serves as a mechanically strong support, allowing archiving of the separation profiles without the need for vacuum gel drying.

The technology described herein can be used to fractionate large proteins, small proteins, acidic proteins, basic proteins and hydrophobic proteins. As an example, large multi-subunit complexes can be fractionated on the surface of a membrane. Hydrophobic integral membrane proteins mobile can be separated, for example, using mobile phases containing high concentrations of organic solvents. The separation technology also can be used to separate electrophoretically silent mutations, wherein proteins and peptides differ only by an uncharged amino acid residue. The separation technology further can be used to fractionate intact proteins.

The methods described herein can involve detection of separated molecules using mass spectrometry. A challenge in the use of matrix-assisted laser desorption/ionization time-of-flight mass spectrometry (MALDI-TOF MS) for the analysis of complex proteomes is the lack of a direct coupling of a highly resolving separation technique with the mass spectrometer itself. Previous work has shown identification of organic compounds using a combination of thin layer chromatography (TLC) and MALDI-TOF MS (see, for example, Ivleva V B, Sapp L M, O'Connor P B, Costello C E. Ganglioside Analysis by Thin-Layer Chromatography Matrix-Assisted Laser Desorption/Ionization Orthogonal Time-of-Flight. Mass Spectrometry. J Am Soc Mass Spectrom. 2005 September; 16(9): 1552-60). Several approaches for coupling TLC to MS that are applicable to the methods described herein include use of MALDI, surface-assisted laser desorption (SALDI), ESI-TOF mass spectrometry, desorption electrospray ionization (DESI) and the development of other TLC-ESI interfaces. For MALDI, the type and concentration of MALDI matrix, as well as the type and composition of solvent to facilitate peptide transport from the inside of the TLC gel to the surface is optimized to allow detection of peptides. Besides direct laser desorption on the untreated plates, small particles can be used as a matrix to couple in the laser power and improve the efficiency of desorption. For example, particle suspension matrices can be successfully utilized for the analysis. Particles of different materials and sizes (e.g. Titania, Graphite or Silicon) can be applied as suspensions to the plates, allowing MS profiles to be recorded directly from them. In another approach, a continuous wave diode laser can be employed as a desorption source. Atmospheric pressure chemical ionization mass spectrometry is used to ionize and subsequently identify the desorbed peptides. A surface sampling electrospray ionization system with tandem MS detection is also suitable for interfacing with MS. The recently discovered method of DESI is an additional approach to coupling with MS. DESI is performed by directing electrosprayed charged droplets and ions of solvent onto the surface to be analyzed. The impact of the charged particles on the surface produces gaseous ions of material originally present on the surface. The resulting mass spectra are similar to normal ESI mass spectra in that they show mainly singly or multiply charged molecular ions of the analytes. The DESI phenomenon can be achieved using both conductive and insulator surfaces. In one or more embodiments, peptide profiles are generated by moving the plate under computer control while directing the stationary DESI emitter charged droplet plume at the plate surface.

The technology described herein can be used, for example, with MALDI-TOF MS for direct analysis of proteins. In this aspect, proteins are fractionated on solid phase supports followed by direct probing with MALDI-TOF laser, such as an orthogonal MALDI-TOF mass spectrometer). Any of a variety of MALDI-TOF MS instruments can be used for performing a method described herein. The prOTOF 2000 MALDI O-TOF (PerkinElmer, Boston, Mass., USA/MDS Sciex, Concord, ON, Canada mass spectrometer is one type of commercially available MS MALDI. This instrument features orthogonal time of flight technology that provides improved instrument stability, resolution, and mass accuracy across a wide mass range compared with conventional linear or axial-based systems. The more accurate and complete protein identification achieved with the prOTOF 2000 reduces the need for peptide sequencing using more complicated tandem mass spectrometry techniques such as Q-TOF and TOF-TOF. In the proOTOF 2000, the MALDI source is decoupled from the TOF analyzer, so that discrepancies arising from the solid phase surface topography or differential ionization of the sample from the surface are reduced before the sample is actually delivered to the detector. The presentation of the proteins bound to a solid phase surface facilitates removal of contaminating buffer species and exposure to protein cleavage reagents (e.g., trypsin) prior to analysis by mass spectrometry. The use of HPLC-based buffers in the fractionation process reduces the potential for downstream interference by detergents and chaotropes during mass spectrometry-based analysis.

If desired, MS analysis of proteins can be performed by providing proteins affixed to solid phase supports for direct probing by a MALDI-TOF laser. Virtual 3D profiles can be generated by separations, providing a charge- and hydrophobicity-based separation profile, followed by desorbing proteins directly from the planar stationary phase using MALDI-TOF mass spectrometry, providing a mass-based separation dimension. Analytical data obtained can be presented as a computer-generated image with 3D map appearance. Such a method can be used, for example, as a starting point for high throughput peptide mass fingerprinting and glycosylation analysis using chemical printing techniques such as piezoelectric pulsing where multiple chemical reactions are conducted on different regions of a spot by defined microdispensing of trypsin in-gel digestion procedures, and allowing peptide mass profiles and characterization of glycosylation, for example, to be achieved from the same spot. A method described herein can be automated, if desired. For example, microdispensing of trypsin and MALDI-TOF matrix solutions can be performed using automated liquid handling equipment.

The technology described herein can be used with mass tagging techniques for differential display proteomics where relative abundances of different proteins in biological specimens are correlated with physiological changes. For example, Isotope-coded affinity tag (ICAT) peptide labeling is one such technique useful for distinguishing between two populations of proteins using isotope ratios. ICAT reagent employs a reactive functionality specific for the thiol group of cysteine residues in proteins and peptides. Two different isotope tags are generated by using linkers that contain either eight hydrogen atoms (do, light reagent) or eight deuterium atoms (d₈, heavy reagent).

A reduced protein mixture from one protein specimen is derivatized with the isotopically light version of the ICAT reagent, while the other reduced protein specimen is derivatized with the isotopically heavy version of the ICAT reagent. Next, the two samples are combined, and proteolytically digested with trypsin or Lys-C to generate peptide fragments. The combined sample can be fractionated using a method for separating a sample comprising a plurality of compounds, as is described herein. The ratio of the isotopic molecular weight peaks that differ by 8 daltons, as revealed by mass spectrometry, provides a measure of the relative amounts of each protein from the original samples.

Other mass tagging approaches include growth of cells in either ¹⁴N- or ¹⁵N-enriched medium, use of regular water (H₂¹⁶O) and heavy water (H₂¹⁸O) as the solvent during Glu-C proteolysis of samples, use of acetate (d₀) and trideuteroacetate (d₃) to acetylate primary amino groups in peptides, methyl esterification of aspartate and glutamate residues using regular methanol (d₀) or trideuteromethanol (d₃), ¹²C and ¹³C labeled tri-alanine peptides iodoacetylated on their N-termini, and use of 1,2-ethanedithiol (d₀) and tetraalkyl deuterated 1,2-ethanedithiol (d₄) to measure differences between O-phosphorylation sites in samples using beta-elimination chemistry.

Similarly, ExacTag technology (PerkinElmer Inc.) can be suitably combined with a method for separating a sample comprising a plurality of compounds, as is described herein. The ExacTag technology is a versatile multiplex protein labeling technology. The ExacTag Labels (Isobaric Protein Labels) are small discreet labels of a proprietary composition. The unique properties of ExacTag Labels allow for the simultaneous measurement of more analytes than any other commercially available system. ExacTag Labels are detectable by MS.

Recently, it has been demonstrated that 2DGE and ICAT labeling technology can be combined into a single differential display platform. Proteins from two different samples are labeled with heavy and light ICAT reagents, combined and then separated by 2DGE. The gel-separated proteins are detected with a sensitive protein stain, excised, treated with protease and identified by peptide mass profiling. Additionally, selected peptides can be evaluated further using collision-induced dissociation (CID) and sequence database searching. One important application of ICAT differential display in 2D gels is for the assessment of the relative abundances of protein isoforms that arise from post-translational modification. In one aspect of the technology described herein, a method for separating a sample comprising a plurality of compounds can be combined with ExacTag labeling into a single platform for highly multiplexed differential display proteomics.

Mass tagging approaches based upon the same basic principles as the ICAT strategy include growth of cells in either ¹⁴N- or ¹⁵N-enriched medium, and the use of regular water (H₂¹⁶O) and heavy water (H₂¹⁸O) as the solvent during Glu-C proteolysis of samples, leading to the incorporation of two 180 or two 160 atoms in the C-terminal moiety of each proteolytic fragment. This results in a 4 dalton difference in mass between paired peptides. Acetate (d₀) and trideuteroacetate (d₃) can be employed to acetylate primary amino groups in peptides. Similarly, methyl esterification of aspartate and glutamate residues using regular methanol (d₀) or trideuteromethanol (d₃) can be used as an isotope tagging strategy. ¹²C and ¹³C labeled tri-alanine peptides iodoacetylated on their N-termini for mass tagging experiments. Finally, 1,2-ethanedithiol (d₀) and tetraalkyl deuterated 1,2-ethanedithiol (d₄) can be employed to measure differences between O-phosphorylation sites in samples using beta-elimination chemistry. The pendant sulfhydryl group is then reacted with biotin iodoacetamidyl-3,6-dioxaoctanediamine. In one aspect of the technology described herein, a method for separating a sample comprising a plurality of compounds can be used with mass tagging technologies as a separation platform for differential analysis of protein expression changes and post-translational modification changes.

The technology described herein can be used with inductively-coupled plasma mass spectrometry (ICP-MS) for the trace elemental analysis of metalloproteins, such as selenoproteins, zinc metalloenzymes, cadmium-binding proteins, cisplatin-binding drug targets, and myoglobins subsequent to fractionation by planar electrochromatography. Laser ablation ICP-MS permits trace element analysis by combining the spatial resolution of an ultraviolet laser beam with the mass resolution and element sensitivity of a modern ICP-MS. UV laser light, produced at a wavelength of 193-266 nm is focused on a sample surface, causing sample ablation. Ablation craters of 15-20 microns are routinely produced by the instrumentation. Special sample preparation is not necessary for the procedure. Ablated material is transported in an argon carrier gas directly to the high temperature inductively-coupled plasma and the resulting ions are then drawn into a mass spectrometer for detection and counting. A mass filter selects particles on the basis of their charge/mass ratio so that only specific isotopes are allowed through the filter and can enter the electron multiplier detector mounted at the end of the mass spectrometer (quadrupole, magnetic sector or time-of-flight instrumentation). Detected signals of individual isotopes can be converted to isotopic ratios or, when standards are measured along with the unknowns, to the actual element concentrations. Laser ablation ICP-MS can be used for directly measuring phosphorous as m/z 31 signal liberated from phosphoproteins on electroblot membranes. Using Laser ablation ICP-MS, 16 pmole of the pentaphosphorylated betα-casein can be detected on polymeric membranes.

A method for separating a sample comprising a plurality of compounds can be used as a platform for the direct analysis of protein phosphorylation, without the use of radiolabels or surrogate dyes. ICP-MS is one example of an analytical technique useful for detecting phosphomolecules when performing a method as described herein. The detection of low concentrations of phosphorous presents certain analytical challenges for ICP-MS due to its poor ionization in the argon ICP and the presence of interfering polyatomic species directly at mass 31 (¹⁵N⁶O and ¹⁴N¹⁶OH) and indirectly at mass 32 (¹⁶O₂ and ³²S). Phosphorous has a high first ionization potential of 10.487 electron volts (Wilbur and McCurdy, 2001). This translates to a poor conversion of phosphorous (P) atoms to P⁺ ions in the inductively coupled plasma. In a well-optimized ICP-MS, this translates to a 6% conversion of P atoms to P⁺ ions, a relatively low response factor for ICP-MS. It is known to one skilled in the art that phosphate groups in proteins and peptides readily bind certain trivalent metal ions, such as aluminum (III), gallium (III) and iron (III). Using ICP-MS, as little as 1 part per billion (ppb) of these metal ions can be detected. The ionized conversion of aluminum, which has a first ionization potential of 5.986 electron volts, is 99% under identical run conditions as described for phosphorous. Thus, detecting aluminum instead of phosphorous improves detection 16-fold. In addition, the specific detection of the trivalent metal ions shifts the detection window away from the described biological background signal. The atomic masses of aluminum, gallium and iron are 26.98, 69.7 and 55.85, respectively. Among these three trivalent metal ions, the ferric ion poses problems due to polyatomic interferences arising from ArN, ArO and ArOH at the interface region of the ICP-MS. Gallium is probably the most suitable metal ion for the proposed application. Both ⁶⁹Ga, and ⁷¹Ga signal can be quantified by the method, minimizing the probability of overlapping signal from other molecular species.

The detection of proteins using ICP-MS-based detection procedure can include the following steps. First, proteins are separated by a method as described in accordance with one aspect of the technology described herein. The planar stationary phases are then incubated with 1 mM gallium chloride, 50 mM sodium acetate, pH 4.5, 50 mM magnesium chloride. Next, the planar stationary phases are washed repeatedly in 50 mM sodium acetate, pH 4.5, 50 mM magnesium chloride to remove excess metal ions. The individual spots on the planar surface are subjected to laser ablation ICP-MS methods where gallium signal is quantified rather than the phosphorous signal. Alternatively, the phosphorous signal can be read without incubating in the gallium solution. Sampling can be performed by single or multi-spot analysis, straight line scans or rastering. To aid in spot selection, the proteins on the planar stationary phase can be stained with a total protein stain, prior to the incubation with the gallium ions.

The technology described herein can be used with protein microarrays for protein expression profiling and studying protein function and for generating protein microarrays. Small planar surfaces can be spotted with a defined mixture of proteins that are subsequently fractionated by a method described herein. Though the constituent proteins are not explicitly assigned a pre-determined coordinate in the resulting orthogonal matrix of spots thus generated, the identities of the spots can simply be determined by mass spectrometry, by immunodetection or by systematic omission of each protein from the mixture in subsequent separations. Once the location of each protein in the profile or subset thereof is known, the array can be used as conventional protein arrays, such as for profiling autoantibody responses in autoimmune disease and screening for other protein-protein, receptor-ligand, enzyme-substrate, enzyme-inhibitor or even protein-DNA interactions. The advantages of the arraying approach are that a dedicated pin-based or piezoelectric spotting device is not necessary and the membrane arrays are amenable to filter-based protein microarray techniques as described recently. For example, a filtration approach that allows multi-stacking of protein chips can be used for simultaneously probing with a particular reagent.

The technology described herein can be used for examination of biomarkers associated with specific proteins present in plasma, urine, lymph, sputum and other biological fluids. Serum albumin in particular is a high abundance blood protein with broad binding capability that serves as a depot and transport protein for numerous exogenous and endogenous circulating compounds. Once plasma is fractionated into its constituent serum protein components using methods described in this technology, peptides associated with discrete proteins, such as albumin, haptoglobin, α₂-macroglobulin or immunoglobulin, can be selectively eluted and identified by MS. The peptides can be acid eluted with 0.2% trifluoroacetic acid and can subsequently be concentrated using reversed phase resin prior to analysis. Using this technique, noncovalently bound peptides can be isolated from a variety of proteins, such as hsp 70, hsp 90 and grp 96. Such a method obviates the need for separating the peptides from the protein by a molecular weight cut-off membrane. Instead, the target protein remains affixed to the planar substrate and the peptides are eluted away from it.

The technology described herein can be used for the fractionation of complex oligosaccharides, glycoproteins, glycolipids, proteoglycans, and oligosaccharides pre-derivatized with fluorophores (such as 8-aminonaphthalene-1,3,6-trisulfonicacid (ANTS) and 2-aminoacridone (AMAC)). Protein glycosylation is used for biochemical alterations associated with malignant transformation and tumorogenesis. Glycosylation changes in human carcinomas contribute to the malignant phenotype observed downstream of certain oncogenic events. Technologies that permit the rapid profiling of glycoconjugate isoforms with respect to oligosaccharide branching, sialyation and sulfation are invaluable tools in assessing the malignant nature of clinical cancer specimens.

A commercial package for conducting a method described herein, such as two-dimensional electrically-driven planar chromatography/thin-layer chromatography, is provided by the technology described herein. The commercial package can include, for example, a planar stationary phase for loading a sample comprising one or more biomolecules, at least one buffer solution, and an instruction booklet outlining instructions on how to use the commercial package for separating a sample containing two or more biomolecules using a method according to one or more embodiments described herein. The commercial package further can include a wick, wherein the wick can be selected from, for example, cellulose-based filter paper, Rayon fiber, buffer-impregnated agarose gel, and moistened paper towel. The commercial package further can include an impermeable barrier to cover the stationary phase, wherein the impermeable barrier is glass plate, polyethylene film or silicone oil.

Although the systems and methods described herein can be used for any charged molecule, the technology is described with reference to the separation of proteins, peptides and glycans. Such description is for convenience only and is not intended to limit the technology. Application of the systems and methods described to other molecules will be apparent from the examples and descriptions which follow.

In the examples that follow, the following model peptides and phosphopeptides from Anaspec Inc., San Jose, Calif., were used. Anaspec Incorporated, San Jose, Calif.: Insulin Receptor peptide (amino acids 1142-1153), Sequence TRDIYETDYYRK [SEQ. ID NO 1], (Catalog # 24537), Kinase Domain of Insulin Receptor, Sequence TRDIYETDpYYRK [SEQ. ID NO 2], (Catalog # 20274), Kinase Domain of Insulin Receptor-2, Sequence TRDIpYETDYYRK [SEQ. ID NO 3], (Catalog #20292), Kinase Domain of Insulin Receptor-4, Sequence TRDIYETDYpYRK [SEQ. ID NO 4], (Catalog #20273), Kinase Domain of Insulin Receptor-5, Sequence TRDIpYETDpYpYRK [SEQ. ID NO 5], (Catalog # 20272), Calcineurin (PP2B) Substrate, Sequence DLDVPIPGRFDRRVSVAAE [SEQ. ID NO 6], (Catalog # 22589), Calcineurin (PP2B) Substrate, Sequence DLDVPIPGRFDRRVpSVAAE [SEQ. ID NO 7], (Catalog # 24516). In addition, another model peptide and phosphopeptide were obtained from SynPep Corporation, Dublin, Calif. The peptides were: p60 c-src peptide (amino acids 521-533) [SEQ. ID NO 8], Sequence TSTEPQYQPGENL [SEQ. ID NO 9], (Catalog #04-10-22-02-PEK), and pp60-src phosphopeptide, Sequence TSTEPQpYQPGENL [SEQ. ID NO [0], (Catalog #04-10-22-03-PEK). Lyophilized bovine α-casein S1 and dephosphorylated bovine α-casein S1; trypsin of proteomics grade quality, sequencing grade trypsin, lyophilized bovine α-casein S1, dephosphorylated bovine α-casein S1, β-casein, ovalbumin, rabbit muscle phosphorylase α, and riboflavin-binding protein were obtained from Sigma Chemical Company, St. Louis, Mo. Human heat shock protein 90 (HSP90) from human HeLa cells was procured from Stressgen Biotechnologies Corporation, Victoria, British Columbia, Canada.

### EXAMPLE 1

### 2D TLC Separation of Peptides

This example describes separation of peptides using TLC in two dimensions. Six model proteins were digested with trypsin and separated as described.

Trypsin (Sigma) was reconstituted in 1 mM HCl to obtain a concentration of 1 mg/mL. Model proteins β-casein and ovalbumin were dissolved in water and 10 mM ammonium carbonate, pH 8.5 buffer respectively. The final concentration in both cases was about 1 mg/mL. The proteins were denatured by heating them to 90° C. Subsequently, the trypsin and the protein were added together in a ratio of 1:10, according to the manufacturer recommended protocol and incubated for a day at 37° C. for 30 minutes. Formic acid (1%) was added to quench the reaction. Separation of the peptides obtained by tryptic digestion of proteins was performed using thin-layer chromatography (TLC) as follows: The separation was performed on HPTLC Cellulose plates or Silica gel 60 glass-backed HPTLC plates (Merck, Darmstadt, Germany). Using either an automated sample applicator or using a capillary bore, total volumes of 1-5 μL were applied as bands or circular spots onto TLC plates. In the case of the applicator, a sample concentration of 1-4 mg/mL was used to deliver the sample at a dosage speed of 30-100 mL/s generating bands with a width of about 3-6 mm. While using the capillary bores, the concentrations were relatively high (4 mg/mL) and the spot size was minimized to obtain a small diameter. In both cases, the sample was applied at about 10 mm from the edge of the plate and after the application, TLC plates were dried under nitrogen stream.

Development of the TLC plates was carried out using the following solvent systems: The mobile phases used in the first and second dimension were 2-butanol/ammonia (25%)/pyridine/water (39/10/2/26, v/v/v/v) and 2-butanol/acetic acid/pyridine/water (30/6/20/24, v/v/v/v) respectively. The dried plate was put in a chamber with the solvent mixture and the solvent front allowed to migrate a distance of about 5 cm, which was achieved in about 45 min. The plate was dried before the second dimension, turned 90° and development in the second dimension was performed. The rate of solvent migration diminishes beyond 5-7 cm in TLC, limiting the plate area utilized for separation. The plate was removed from the chamber after the second dimension and was once again dried under nitrogen and sprayed with ninhydrin (0.5% in 2-propanol) or fluorescamine (0.2% in acetone).

2D TLC separation of a tryptic peptide digest of P-casein is shown in FIG. 1. TLC separation was achieved using either the acidic or basic solutions without significantly affecting the resolution or overall peptide profile. However, the separation is not completely orthogonal, as migration in the first dimension correlates somewhat with migration in the second dimension. This is evident as peptide spots resulting from the 2D TLC separation were largely distributed along a diagonal on the plate.

### EXAMPLE 2

### 2D TLE/TLC Separation of Peptides

This example describes two dimension separation of peptides using thin-layer electrophoresis (TLE) and TLC. 2D TLE/TLC separation of peptide digests was accomplished by the well-established HTLE method. Briefly, 88% formic acid/glacial acetic acid/water, pH 1.9 (50:156:1794 v/v/v) was used as the mobile phase for the TLE dimension and n-butanol/pyridine/glacial acetic acid/water (75/50/15/60 v/v/v/v) was employed for the TLC dimension. TLE separations were performed using a Hunter Thin-Layer Electrophoresis (HTLE) instrument, Model HTLE-7002, CBS Scientific, Del Mar, Calif. Cellulose HPTLC plates were used for the separation.

FIG. 2 portrays a typical 2D separation of a O-casein tryptic peptide digest using a combination of TLE and TLC. Reasonable resolution separations of peptides are achieved by the TLE/TLC method. However, the separation results in a relatively low number of detected spots. Also, no anodically migrating peptides were detected.

### EXAMPLE 3

### Two Dimensional (2D) PEC/TLC Separation of β-Casein and Ovalbumin Tryptic Peptide Digests

Tryptic peptide digests of ovalbumin and β-casein were performed as described in Example 1. Separations of tryptic digests of α-casein and ovalbumin were performed as described below.

The HTLE apparatus for PEC/TLE was assembled as per the manufacturer's instructions (CBS Scientific). Each of the buffer tanks was filled with approximately 500 mL of the buffer. Whatman 3 mM paper, folded in the middle, was used as a wick to transfer the solution from the buffer tanks (lined with platinum electrodes across the length of the tank) onto the separation plates. Wicks were made by cutting the paper to fit and overlap the TLC plate by about a cm from the edge of the plate. The wicks were then wetted in a glass tray, excess solvent drained by tapping on the sides of the tray and immersed in the buffer tanks on one edge leaving the other edge to overlap the plate. A pressure of about 0.7 Atm (10 PSI) was applied using the bladder and clamp setup of the HTLE instrument to pressurize and eliminate excess solvent. The pressure was removed momentarily to place the plates ready for separation (described below) and constant pressurization was maintained during the electrically-driven separation. The platinum wires on the tanks, located on either side of the unit, connected to a power supply and the base, where the plate is located, were maintained at ambient temperature (˜25° C.) with a circulating water bath.

Silica gel 60 plastic-backed TLC plates or HPTLC Cellulose plates were used for the separation. A ‘+’ (plus) mark, indicating the location of the sample (henceforth referred to as ‘the origin’), was applied, using a common #2 (or HB) grade graphite pencil, about 5 cm from a corner of the plate. At acidic/low buffer pH this corner should be located closer to the anode as most of the peptides migrate towards the cathode in the presence of an electrical field. 3-5 μL of the peptide digest was applied onto the plates in 1 μL aliquots, followed by drying under nitrogen stream. Plates were then pre-wetted with the solvent/buffer system to be used for the separation. Whatman 3 mM paper was wetted in the solvent mixture; excess solvent removed by tapping against another paper and was used to wet the plate surface. Care was taken to ensure minimal and uniform wetting and excess solvent was removed by gently pressing a dry paper on the plate. The area around the origin was wetted by overlaying a wet paper having a hole slightly larger than the spot size onto the plate. The paper was gently pressed from all the sides so that the solvent was directed towards the center of the spot, thereby concentrating the sample (and avoiding any sample spreading). Finally, the HTLE setup was dismantled, excess solvent wiped off the surface and the spotted plate was placed with overlapping wicks and pressurization, ready for separation.

HTLE/PEC in the First Dimension:

Separation of the 1-5 μL peptide spot on the Silica gel 60 or Cellulose HPTLC plate was achieved with pH 4.7 buffer (n-butanol/pyridine/glacial acetic acid/water, 50/25/25/900, v/v/v/v) in the first dimension. A potential of 300-400 V was applied across the plate, generating a current output of 20 mA in this setup, which was current limited. A constant pressure of 0.7 Atm (10 PSI) was applied to the plate surface and the plate was cooled using a water circulator from beneath to prevent excessive heating due to the applied potential (Joule heating).

TLC in the Second Dimension:

Thin-layer plates from the first PEC dimension were allowed to dry thoroughly under a stream of nitrogen and introduced into a TLC chamber containing the solvent mixture for the separation in the second dimension. Two solvent systems used in the previously described TLC separations were found to be suitable, with increased resolution in the PEC/TLC mode relative to when only TLC was used in both the separation dimensions. These were 2-butanol/ammonia (25%)/pyridine/water (39/10/2/26, v/v/v/v) and 2-butanol/acetic acid/pyridine/water (30/6/20/24, v/v/v/v). Plates were either air dried or dried under nitrogen stream after the second dimension separation.

For MALDI-TOF MS and tandem MS experiments using the QSTAR instrument, ID PEC separation of the tryptic digest of bovine α-casein was performed as follows. Two 5 mm long bands of peptide digest were loaded on the Silica gel 60 glass-backed HPTLC plate, each band containing 1.5 μg of bovine α-casein peptide digest. Separation was achieved using the standard PEC experimental conditions as described above, but only for 25 min duration, in order to generate separations scaled appropriately for the QSTAR MALDI sample plate.

Total peptide staining was achieved by spraying 0.05% (w/v) fluorescamine in acetone directly onto the dried separation plate. No destaining was necessary as fluorescence is exhibited only upon covalent binding of the dye to the N termini and ε-amino groups of the peptides. In the case of the phospho-specific dye, Pro-Q Diamond Blot Stain (Invitrogen, Eugene, Oreg.), spray staining was performed following the fluorescamine visualization and drying process. The dye was diluted/reconstituted using the appropriate buffer, according to the manufacturer's instructions. Destaining prior to fluorescence imaging was performed by quickly rinsing in a solution of 7% acetic acid and 10% ethanol (v/v) and then air drying. Excessive destaining led to elution of the peptides from the chromatographic plates.

Visualization of the separated peptide spots was performed using either a ProXPRESS™ classic or ProXPRESS 2D Imager (PerkinElmer, Boston, Mass.) operating in the fluorescence mode. Briefly, all images were acquired with 16-bit grey scale depth. Fluorescent stains were imaged using the top illumination mode of the instrument. Various CCD camera exposure times were investigated in order to obtain images with the highest contrast and minimal background signal. The excitation and emission wavelength filter settings for fluorescamine were 390/70 and 480/30 nm, respectively. The CCD camera exposure time was typically one second. In the case of Pro-Q Diamond stain—excitation and emission wavelength filter settings were 540/25 and 650/150 nm, respectively, with exposure times of 10-30 seconds. No flat-field subtraction was performed. Images were exported both in the default instrument format and as JPEG or TIFF graphical file formats.

In the case of ID PEC separated tryptic digest for QSTAR MALDI-TOF MS analysis, only peptide spots in the upper lane on the plate were visualized with fluorescamine solution while the unstained peptide spots in the lower lane of the plate were marked and labeled using the image of the upper lane as a guide. The glass HPTLE Si 60 plate treated with αCHCA was glued on an Aluminum sheet with thickness of about 0.15 mm, identical in size to the QSTAR MALDI-o-TOF sample plate. The total thickness of the TLC plate mounted on the aluminum sheet was targeted to match the original sample plate thickness for optimal laser focusing. The obtained assembly was mounted directly on the holding frame of the standard QSTAR sample plate and was loaded into the vacuum chamber of the MALDI-o-TOF MS instrument.

Images of separated tryptic peptides from α-casein and dephosphorylated α-casein were matched using the image analysis software, ProFINDER 2D (PerkinElmer). An overlay of the two images was created with the software's warping function activated, so that matched spots were overlaid upon one another. Matching was facilitated by manually assigning several landmark or anchor spots to the two images. With the selected display mode, spots in green or magenta are either unmatched or show substantial differences in quantity and those in black match and are present at similar levels. The green spots were assigned to the α-casein profile, while the magenta spots were assigned to the dephosphorylated α-casein profile.

Upon using either of the described TLC solvent systems in the second dimension, the peptide profile was orthogonal with respect to the PEC separation. Correlation between the two separation modes was not discernible and consequently spots were not distributed along a diagonal line. This is illustrated in FIG. 3, showing separation of a β-casein tryptic peptide digest as described above.

### EXAMPLE 4

### Two Dimensional (2D) PEC/PEC Separation of β-casein and Ovalbumin Tryptic Peptide Digests

Tryptic peptide digests of ovalbumin and P-casein were performed as described in Example 1. Separations of tryptic digests of β-casein and ovalbumin were performed as described in Example 3, except that the second dimension separation was a second PEC. On some plates, where a second dimension of PEC was performed, the plate was turned 90° from the initial position and subjected to the electrically driven separation (2D) in a similar manner, but using an alternative buffer (88% formic acid/glacial acetic acid/water, pH 1.9 (50/156/1794 v/v/v). However, as with the 2D TLC approach, this was found not to produce completely orthogonal peptide separations.

### EXAMPLE 5

### Orthogonal MALDI TOF MS of Peptides Separated Using Orthogonal PEC and TLC

Peptides separated as described in Example 3 were analyzed by orthogonal MALDI TOF MS. The separation plate was superimposed over a print out of its fluorescamine-generated fluorescent image (total peptide spots), obtained from the ProXPRESS 2D Imager, and the spots were marked using a pencil. The plastic-backed plate was then cut to fit the size of the PrOTOF™ gold-coated, glass-backed MALDI target plates (PerkinElmer, Boston, Mass.), where they were affixed using double sided adhesive tape. In the case of 1D separation, it was possible to align the spots along the ‘prefixed’ well positions on the plate and the spots were easier to locate. 2 μL of 5 mg/mL, αCHCA matrix, reconstituted in 50% acetonitrile and 0.1% trifluoroacetic acid, was cast on top of the circled spots, based on their locations as seen from the fluorescence image template. The plates were allowed to air dry before being introduced into the prOTOF 2000 MALDI-o-TOF MS instrument using orthogonal ion extraction technology (PerkinElmer/Sciex, Boston, Mass.). Control spots on Silica gel 60 plastic-backed TLC plates, without the matrix, did not yield any detectable mass spectra. The PrOTOF instrument is equipped with a CCD camera and a CRT monitor was attached to precisely locate the marked spots. Laser shots at 75% energy were directed at the center of these spots, mass spectra corresponding to the spots were obtained and the data files saved. The m/z value of the precursor ions and known fragments in the spectra in the case of P-casein and ovalbumin digests, separated on Silica gel 60 plastic-backed TLC plates, were compared to the known standards from the literature. The manual spot location and thereby analysis in the case of 2D-resolved peptides on the plates was more challenging, but essentially accomplished in the same manner as the 1D separations. The image of the fluorescently-derivatized peptides was used as a template to locate peptides on the plate.

In the case of the ID PEC separated tryptic digest, only peptide spots in the upper lane on the plate were visualized with fluorescamine solution while the unstained peptide spots in the lower lane of the plate were marked and labeled using the image of the upper lane as a guide. α-CHCA MALDI-TOF matrix was prepared as 5 mg/ml solution in 50% acetonitrile containing 1% formic acid. The α-CHCA MALDI-TOF matrix was applied to the traced spots once, 2 μl of the corresponding matrix solution per spot. The dried Silica gel 60 glass-backed HPTLC plate treated with α-CHCA was glued on to an aluminum sheet with thickness of about 0.15 mm, identical in size to the QSTAR MALDI sample plate. The total thickness of the TLC plate mounted on the aluminum sheet was targeted to match the original QSTAR MALDI sample plate thickness for optimal laser focusing. The obtained assembly was mounted directly on the holding frame of the standard QSTAR instrument with oMALDI (orthogonal Matrix-assisted Laser Desorption/Ionization) ion source (Applied Biosystems/Sciex, Foster City, Calif.) and was loaded into the vacuum ion-source chamber of the QSTAR instrument.

MS and MS/MS studies of the 1D PEC-separated peaks from Silica gel 60 glass-backed HPTLC plates was performed using a QSTAR pulsar I tandem mass spectrometer consisting of a reflectron time-of-flight (TOF) analyzer as well as three quadrupoles, i.e. ion guide Q0, mass filter Q1, and collision cell Q2. The 384-well grid was used to manually navigate the QSTAR MALDI sample plate in order to facilitate visual inspection of the labels and recognition of the peptide peaks in the lower unstained lane of the TLC plate. The MALDI-TOF MS and MS/MS spectra were acquired in the positive ion mode, each spectrum being the average of 174 and 300 individual scans, respectively. The mass axis of the instrument was calibrated using the α-CHCA mass ion at 190.0504 a.m.u and angiotensin I mass ion at 1296.6853 a.m.u. The samples were irradiated with an N₂ laser, emitting at 337 nm, with pulse rate of 20 Hz and pulse energy of 13.5 μl to optimize the signal to noise ratio. For each labeled spot, collection of a survey MS spectrum was followed by selection of a few intense ion-mass peaks for product ion MS/MS scan experiments. The parent ion, mass-selected by mass filter Q1, underwent collision induced dissociation (CID) in the collision cell Q2, and the resultant fragment or daughter ions were pulsed into the TOF analyzer. The fragment ions were mass-separated in the TOF analyzer and detected by a dual microchannel plate (MCP) detector with 4 Anode detectors. In the experiments, the TOF mirror voltage was 997 Volt; the TOF plate voltage was 359 Volt; the TOF grid voltage was 471 Volt and the TOF linear voltage was 4 kVolt. The acquired MS/MS spectra were analyzed for peptide sequencing and identification using the MASCOT database search engine (www.matrixscience.com) and the SwissProt protein database. When a neutral loss was observed in a peptide MS spectrum, the variable modification of phosphorylation for phospho-serine-carrying and/or phospho-threonine-carrying peptides was included in the database searching parameters.

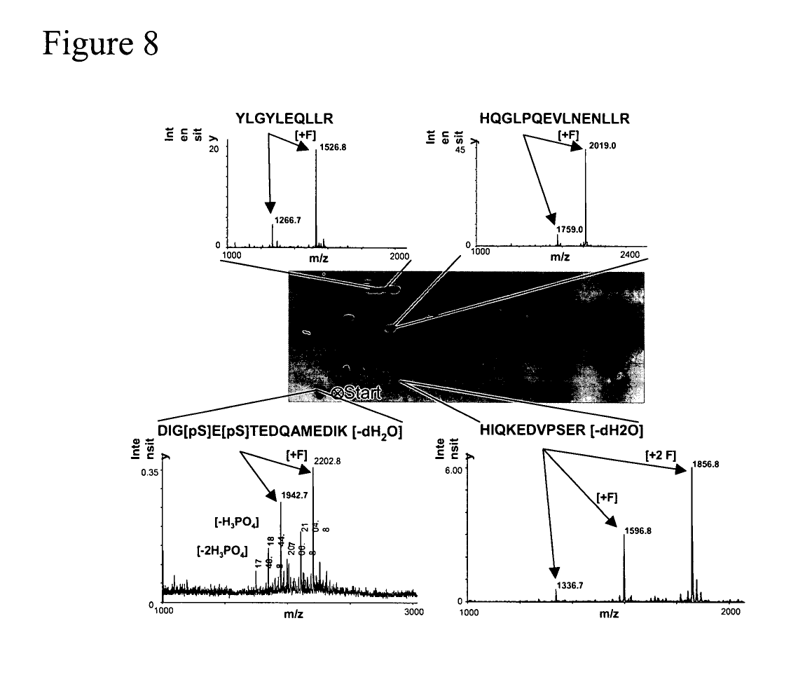

FIG. 8 displays the image of a separated tryptic digest of bovine α-casein S1 and four representative (out of a total of fifty) analyzed MALDI-TOF spectra of the peptide peaks. The fluorescamine-stained image of the separated peptide spots is shown in the middle. Peptide identification presented in the figure was based on the match of MALDI-TOF MS-detected masses to those in the theoretical tryptic digest of the protein. The results demonstrate that MALDI-TOF MS analysis of separated peptides on HPTLC plastic-backed silica plates results in a high accuracy of ion mass determination and thus enables identification of the peptides. The major peptide peaks correspond to the fluorescamine-modified peptides (marked as [+F] or [+2F] for 1 or 2 fluorescamine modifications, respectively) although the unmodified peptides can also be seen. FIG. 8 also demonstrates that the phosphopeptide was separated from the bulk of the peptides in the digest, appearing anodically relative to the origin. Loss of phosphate groups during the MALDI acquisition was also used for identification of the phosphorylated peptides (lower left spectrum). In the second dimension, the phosphopeptide does not migrate due to its high hydrophilicity. Consequently, the phosphopeptide is easily located based upon its unusual migration relative to all other peptides in the digest. Other modifications such as loss of water, acetylation, monovalent metal ion (Na⁺, K⁺, Li⁺) complexes were also observed (data not shown).

### EXAMPLE 6

### Preparing Monolithic Sol-Gel Based Titania Surfaces for Separation of Proteins and Peptides

Sol-gel technology has been used to obtain monolithic silica coatings on TLC supports, as well as for other applications that include coating of columns as well as capillaries for use in HPLC, CEC and other purification/separation purposes. With respect to TLC, sol-gel coated silica plates have been manufactured and marketed by Merck (Darmstadt, Germany). These plates are 10 micron thick, leading to a smaller loading capacity, and further lack the ˜300

A pore sizes necessary for protein separations. Surfaces with titania coatings also can be useful for protein/peptide separations. Titania-based coatings have been known to generate bi-directional electroosmotic flow (EOF) making them viable as PEC supports. Under certain circumstances, titania selectively binds to phospho proteins/peptides. In addition, titania has stability to pH changes and its mechanical strength is comparable to silica. Thus, titania-based supports can be used for simultaneously separating can be coupled to MALDI TOF MS detection, as organic matrix-free UV-absorbing materials, reducing or eliminating multiple steps needed in applying conventional organic matrices, as described in Example 3.

Coating layers of titania on planar fused silica substrate can be performed as follows. A fused silica sheet/plate (10 cm×7 cm, optimum thickness, BES optics, UV transmitting so that it can be used as a MALDI target) is thoroughly cleaned and is dipped-into 1 M KOH for several minutes to one hour to expose maximum number of silanol groups on the surface. It is subsequently cleaned in water for 30 min. in 0.1 M HCl and dried. The sol solution is prepared as follows. Vortex the following in a 2 mL polypropylene centrifuge tube:

Sol-active organic component (hydroxyl terminated poly(dimethyl siloxane) PDMS 50 mg.

A sol-gel precursor Titanium (IV) isopropoxide, 50 uL

Two solvents: methylene chloride and 1-butanol, 200 uL each

A mixture of two surface deactivation reagents—1,1,1,3,3,3-hexamethyldisilane (HDMS, 8 uL) and poly(methylhydrosiloxane (PHMS, 2 uL)

Sol-gel chelating agent, 27% TFA in water—18 uL

The contents of the tube are centrifuged at 13,000 rpm (15,682×g). Clear solution from the top of the tube is transferred to another clean vial by decanting and used to coat the fused silica plate. The above sol solution is placed on the fused silica plate (immersed in a seal-proof bag) and left for 15-30 minutes to facilitate the creation of a surface-bonded coating due to sol-gel reactions taking place in the coating solution located on the surface of the plate. This can be repeated until the desired thickness is obtained. Ideally, the plate can be dipped in a tray/beaker containing the solution repeatedly (layer-by-layer).

The plate is placed in a desiccator for 12 hours and then conditioned at 250-300 degrees C. under nitrogen for 3 hrs. This way, a layer with thickness of roughly 70-75 um can be produced, with pore sizes of 300 angstrom to one micron. The titania-coated fused silica plate is then subjected to separation in a manner analogous to example 2.

### EXAMPLE 7

### PEC/TLC of Phosphopeptides

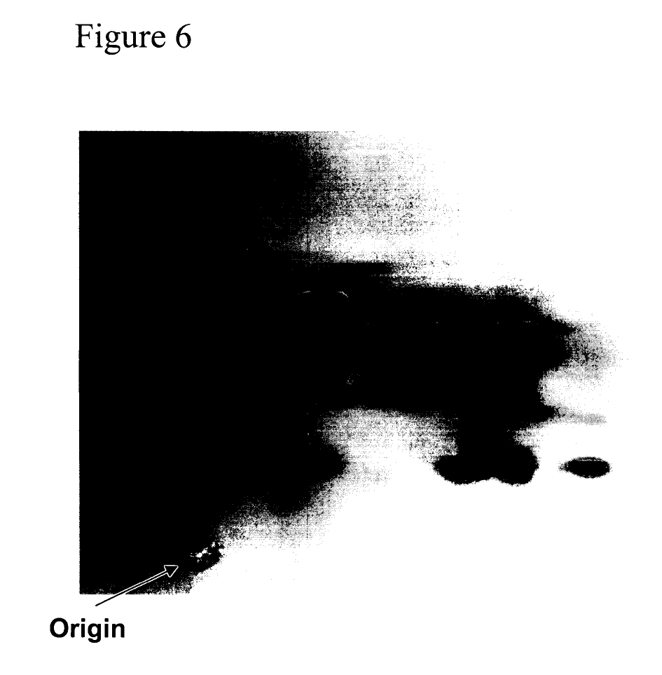

To further document the utility of the 2D separation for highlighting phosphopeptides, separation of tryptically digested α-casein and dephosphorylated α-casein was performed. Differences in the peptide spots for these two separated protein digests directly identified the separated phosphopeptides as the dephosphorylated variant, as the variant obtained via phosphatase treatment of the α-casein had fewer phosphopeptides. The manufacturer reports that 1-2 phosphate residues remain on the phosphatase-treated α-casein and that the phosphorylated variant contains 7-8 phosphorylated residues. After the separation in both dimensions, peptides were visualized by spraying the plates with fluorescamine prepared in acetone, as described previously. Images thus obtained were overlaid for comparison using the PROFINDER software (PerkinElmer). FIG. 6 shows the two individual images overlaid with the warping feature selected. Warping allows matching spots from the two images to be superimposed. The spots from the α-casein image are shown in green and those from the dephosphorylated α-casein image are shown in magenta. Matching spots present at similar intensities appear as black and unmatched spots appear in their respective colors. Spots showing changes appear as a darker shade of the dominant color. FIG. 6 clearly shows that two of the dominant spots (green), one at the origin and one to its left, do not appear in the dephosphorylated α-casein profile. In addition, corresponding hypophosphorylated peptides from the phosphatase-treated α-casein appear as magenta spots in the overlay profile, one to the left of the origin and one to the right of the origin. Both of these peptides are less hydrophilic than their fully phosphorylated counterparts, as demonstrated by their greater migration in the TLC dimension. The observed migration trend is in agreement with the data obtained for model phosphorylated peptides (FIG. 5).

Phosphopeptides can be visualized on the same plates after fluorescamine staining using a phosphorylation-selective fluorescent stain, Pro-Q Diamond phosphoprotein blot stain.

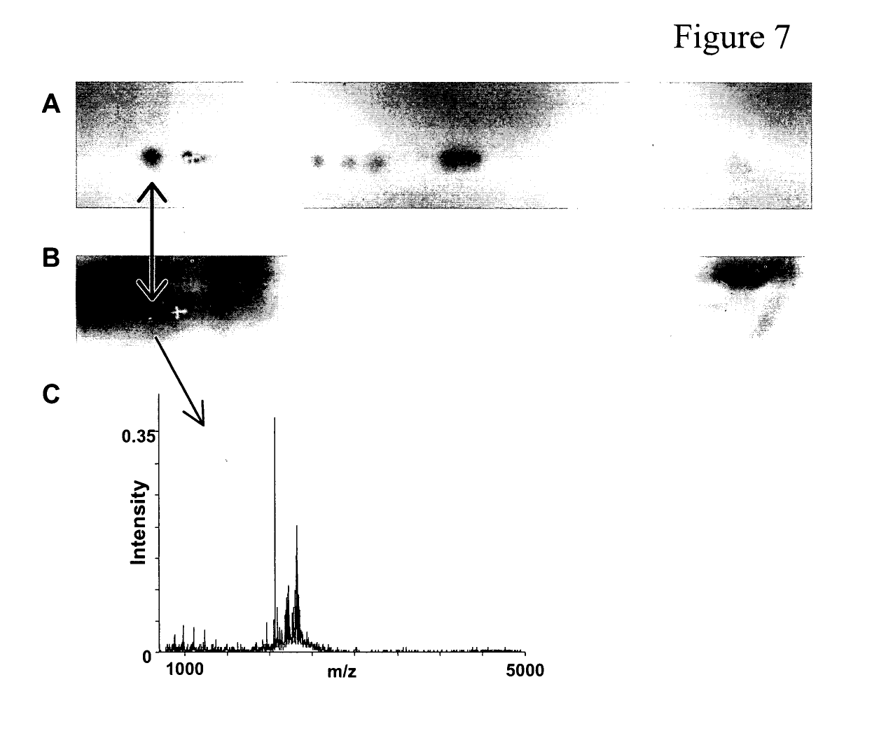

FIG. 7 shows total protein staining using fluorescamine label (FIG. 7A) and, phospho-selective staining using Pro-Q Diamond dye (FIG. 7B), the latter staining only the peptide spot that is located to the left of the origin, clearly indicating the phosphopeptide that has moved to the anode during the 1^{st} dimension PEC separation. It is worth mentioning that due to the difficulties associated with the staining and de-staining protocols of Pro-Q Diamond dye, the peptides had a tendency to be washed away during staining, so a higher affinity phosphorylation-selective dye would improve this procedure substantially.

The identity of the phosphopeptides in the tryptic digest of bovine α-casein was established using MALDI-TOF MS analysis performed directly on the HPTLC plate after the 2D separation, followed by fluorescamine staining. FIG. 7C shows a typical MALDI-TOF MS profile of prominent phosphopeptides, detected from a tryptic digest of P-casein after separation by orthogonal PEC and TLC (Residues 33-48, FQpSEEQQQTEDELQDK [SEQ ID NO [1], Mass: 2061.82 a.m.u., calculated pI: 3.29; residues 16-40, RELEELNVPGEIVEpSLpSpSpSEESITR [SEQ ID NO [2], Mass: 3122.27 a.m.u., calculated pI: 2.51). In addition, peptides covalently modified with fluorescamine during fluorescence-based detection of the spot were found. For instance, the peak centered around a mass of 2339 a.m.u. is attributed to the fluorescamine-derivatization (addition of 278 a.m.u.) of the 2061.82 peptide. The phosphopeptides were separated from the bulk of the peptides in the digest, as they migrated from the origin towards the anode relative. In the second dimension, the phosphopeptides did not migrate due to their high hydrophilicity. Consequently, the phosphopeptides were easily located based upon their unusual migration relative to all other peptides in the digest. Though visualized with fluorescamine, the majority of the peptides in the spot corresponded to the unmodified masses of the peptides.

The many features and advantages of the technology are apparent from the description herein, thus, it is intended to cover all such features and advantages of the technology which fall within the true spirit and scope of the technology. Further, since numerous modifications and variations will readily occur to those skilled in the art, it is not desired to limit the technology to the exact construction and operation illustrated and described, and accordingly, all suitable modifications and equivalents fall within the scope of the technology. It is understood that a variety of molecules, in addition to “biomolecules” can be separated and/or detected using the technology described herein. Thus, use of the word biomolecule throughout this application is intended as an example of a type of molecule that can be present in a sample to be analyzed using the methods described herein.

What is claimed is:

1. A method for separating a sample comprising a plurality of compounds, comprising:
loading a sample onto a planar stationary phase, said sample comprising a plurality of compounds to be separated; and
while retaining the sample on the planar stationary phase,
subjecting the sample to planar electrochromatographic separation in a first direction using a first mobile phase; and
subjecting the sample to a chromatographically-based separation in a second direction using a second mobile phase.

2. The method of claim 1, wherein the compound is selected from the group consisting of proteins, peptides, amino acids, oligosaccharides, glycans and small drug molecules.

3. The method of claim 1, wherein the planar stationary phase comprises an amphiphilic separation medium.

4. The method of claim 3, wherein the amphiphilic separation medium comprises a hydrophobic polymer derivatized with ionic groups.

5. The method of claim 4, wherein the ionic group is selected from one or more of sulfonic acid, sulfopropyl, carboxymethyl, phosphate, diethylaminoethyl, diethylmethylaminoethyl, allylamine and quartenary ammonium residues.

6. The method of claim 4, wherein said hydrophobic polymer is selected from the group consisting of polyvinylidine difluoride, polytetrafluoroethylene, poly(methyl methacrylate), polystyrene, polyethylene, polyester, polyurethane, polypropylene, nylon and polychlorotrifluoroethylene.

7. The method of claim 1, wherein the planar stationary phase is selected from the group consisting of silica based normal and reverse-phase thin layer chromatography resin derivatized with alkyl groups or aromatic groups.

8. The method of claim 1, wherein planar electrochromatographic separation is substantially orthogonal to thin-layer chromatographic separation.

9. The method of claim 1, wherein planar electrochromatographic separation comprises applying a voltage across the planar stationary support in contact with a first liquid mobile phase, causing at least one compound of the sample to be displaced in a first direction along the length of the planar stationary phase, wherein the compounds are separated by the partitioning effect between the liquid mobile phase and the solid stationary phase.

10. The method of claim 9, wherein the separation occurs under electroosmotic forces.

11. The method of claim 9, wherein separation occurs under electrokinetic forces.

12. The method of claim 1, wherein the chromatographically-based separation comprises thin-layer chromatography, wherein the second mobile phase is advanced by capillary action in a second direction along the planar stationary phase, wherein the compounds are separated by the partitioning effect between the liquid mobile phase and the solid stationary phase.

13. The method of claim 1, wherein the chromatographically-based separation is selected from the group consisting zone refocusing planar chromatography and forced flow planar chromatography.

14. The method of claim 1, wherein one or more properties of the first and second mobile phases is different.

15. The method of claim 14, wherein said properties are selected from the group consisting of pH, ionic strength, organic modulator composition and organic content.

16. The method of claim 1, wherein the compounds comprise peptides.

17. The method of claim 16, wherein the peptides comprise phosphopeptides.

18. The method of claim 17, wherein during planar electrochromatographic separation the mobile phase induces migration of phosphopeptides in an opposition direction as an unphosphorylated peptide.

19. The method of claim 18, wherein the mobile phase comprises a mixture of 1-butanol, pyridine, glacial acetic acid and water.

20. The method of claim 17, wherein phosphopeptides are segregated to a preselected region of the planar stationary phase upon separation.

21. The method of claim 1, wherein the planar electrochromatographic separation is done before the chromatographically-based separation.

22. The method of claim 21, further comprising removing the first mobile phase before performing said chromatographically-based separation.

23. The method of claim 1, wherein the chromatographically-based separation is done before the planar electrochromatographic separation.

24. The method of claim 1, further comprising removing the second mobile phase before performing said planar electrochromatographic separation.

25. The method of claim 1, further comprising detecting the separated compounds.

26. The method of claim 25, wherein the detecting comprises a method selected from the group consisting of fluorescence, mass spectrometry, chemiluminescence, radioactivity, evanescent wave, label-free mass detection, optical absorption and reflection.

27. The method of claim 25, wherein the compounds are labeled with a detection agent prior to separation.

28. The method of claim 25, wherein the compounds are labeled with a detection agent after separation.

29. The method of claim 28, wherein said detection agent is selected from the group consisting of colored dyes, fluorescent dyes, chemiluminescent dyes, biotinylated labels, radioactive labels, affinity labels, mass tags, and enzymes.

30. The method of claim 1, further comprising subjecting the sample to analysis in a third dimension using mass spectrometry.

31. The method of claim 30, wherein mass spectrometry is selected from a group consisting of MALDI-TOF mass spectrometry, ESI-TOF mass spectrometry and inductively-coupled plasma mass spectrometry.

32. The method of claim 30, further comprising mass tagging said compounds for differential analysis of protein expression changes and post-translational modification changes.

33. A kit for conducting electrochromatography, the kit comprising:
a separations plate comprising a planar amphiphilic separation medium for loading a sample comprising one or more compounds;
at least one electrode buffer solution suitable for use in planar electrochromatographic separations;
a MALDI-detectable detecting agent.

34. A kit for conducting electrochromatography, the kit comprising:
a separations plate comprising a planar amphiphilic separation medium for separating a sample comprising one or more compounds, and at least one electrode buffer solution suitable for use in planar electrochromatographic separations.

35. The kit of claim 33, further comprising an impermeable barrier to cover said separation medium, wherein said impermeable barrier is glass plate or silicone oil.

36. The kit of claim 33, wherein the separations plate comprises a silica TLC plate.

37. The kit of claim 33, wherein the mobile phase comprises a composition capable to induce migration of phosphopeptides in a direction opposite that of an unphosphorylated peptide.

38. The kit of claim 37, wherein the mobile phase comprises 1-butanol, pyridine, glacial acetic acid and water.

39. The kit of claim 33, wherein the amphiphilic separation medium comprises a hydrophobic polymer derivatized with ionic groups.

40. The kit of claim 39, wherein the ionic group is selected from one or more of sulfonic acid, sulfopropyl, carboxymethyl, phosphate, diethylaminoethyl, diethylmethylaminoethyl, allylamine and quartenary ammonium residues.

41. The kit of claim 39, wherein said hydrophobic polymer is selected from the group consisting of polyvinylidine difluoride, polytetrafluoroethylene, poly(methyl methacrylate), polystyrene, polyethylene, polyester, polyurethane, polypropylene, nylon and polychlorotrifluoroethylene.

42. The kit of claim 33, wherein the planar stationary phase comprises a silica based thin layer chromatography resin selected from the group consisting of normal and reverse-phase derivatized with alkyl groups or aromatic groups.

43. The kit of claim 33, further comprising a wick, wherein the wick can be selected from the group consisting of cellulose-based filter paper, Rayon fiber, buffer-impregnated agarose gel, and moistened paper towel.

44. A method for separating phosphoproteins in a sample, comprising:
loading a sample onto a planar stationary phase at a loading origin, said sample comprising a at least one phosphoprotein; and
while retaining the sample on the planar stationary phase, subjecting the sample to planar electrochromatographic separation in a selected direction;
subjecting the sample to a chromatographically-based separation in a direction other than that of the direction used for planar electrochromatographic separation; and
interrogating a preselected region of the planar stationary phase for the presences of phosphopeptides.

45. The method of claim 44, wherein the preselected region is at or near the loading origin.

46. The method of claim 44, wherein the preselected region is anodically located relative to the loading origin.
